(19) United States
(12) Patent Application Publication
(10) Pub. No.: US 2022/0387621 A1
(43) Pub. Date: Dec. 8, 2022

(54) TARGETED DELIVERY OF NANOCARRIER-CONJUGATED DOXORUBICIN

(71) Applicants: Roger LEBLANC, Miami, FL (US); Jonathan SCHATZ, Miami, FL (US); Piumi LIYANAGE, Miami, FL (US); Artavazd ARUMOV, Miami, FL (US); UNIVERSITY OF MIAMI, Miami, FL (US)

(72) Inventors: Roger LEBLANC, Miami, FL (US); Jonathan SCHATZ, Miami, FL (US); Piumi LIYANAGE, Miami, FL (US); Artavazd ARUMOV, Miami, FL (US)

(21) Appl. No.: 17/774,892

(22) PCT Filed: Nov. 6, 2020
(86) PCT No.: PCT/US2020/059465

§ 371 Date: May 6, 2022

Related U.S. Application Data
(60) Provisional application No. 62/931,594, filed on Nov. 6, 2019.

(51) Int. Cl. A61K 47/69; A61K 47/68; A61P 35/00; A61K 39/395; A61K 31/664; A61K 31/439; A61K 31/573

(52) U.S. Cl. CPC A61K 47/6929; A61K 47/6809; A61K 47/6849; A61P 35/00; A61K 39/3955; A61K 31/664; A61K 31/439; A61K 31/573

(57) ABSTRACT

This disclosure relates generally to compositions of carbon dots, doxorubicin, and transferrin and methods for use of the same in the treatment of DLBCL tumors.

### PRIORITY STATEMENT

This application claims the benefit of U.S. provisional application Ser. No. 62/931,594, filed on Nov. 6, 2019, which is incorporated by reference herein in its entirety.

### BACKGROUND

### Field of the Disclosure

This disclosure relates generally to compositions and method of carbon-nitride dot nanocarrier delivery of chemotherapy agents. More specifically, the disclosure provides carbon nitride dots with doxorubicin and transferrin (CDT) compositions. The disclosure also provides methods for using the compositions in targeted delivery for treatment of blood and circulating cancers, specifically diffuse large B-cell lymphoma (DLBCL) tumors. The CDT compositions exploit TFR1-mediated endocytosis to gain entry into cancer cells. Also provided is a R-CHOP (rituximab, cyclophosphamide, doxorubicin, vincristine and prednisone) composition comprised of CDT.

### Technical Background

Diffuse large B-cell lymphoma (DLBCL) is the most common type of non-Hodgkin lymphoma. The lymphoma is an aggressive cancer that develops from the B-cells in the lymphatic system and is characterized by fast-growing lumps in the neck, armpit, or groin. As such the condition requires rapid treatment.

Common treatments for DLBCL include targeted therapy and standard chemotherapy that is maintained for numerous months. Chemotherapy often includes a regimen of cyclophosphamide, doxorubicin, vincristine, and prednisone, commonly referred to as CHOP. In addition, inclusion of the monoclonal antibody rituximab is a common therapeutic adjunct. Alternative therapies include radiation, and stem cell transplants.

Despite these treatment regimens only about 50% of all patients with DLBCL are cured. Disease stage and International Prognostic Index (IPI) scores correlate with cure rate, with early stage detection and low IPI scores associated with increased survival rates. As such, new treatments are needed to address persistent unmet DLBCL clinical needs.

Carbon-nitride dots (CNDs) are an emerging nanoparticle technology with excellent in vivo stability and distribution and adaptable to covalent conjugation with multiple substrates. CNDs have excellent optical properties with a unique heteroatom structure wherein the functional groups allow for use in a wide range of applications. CNDs for biomedical applications have high quantum yields, good stability, low cytotoxicity, the ability to enter the cell and deliver therapeutic payloads, and then undergo lysosomal degradation. As such CNDs are valuable delivery vehicles for targeted chemotherapy.

Consistent with this, overexpression of transferrin receptor (TFR1) is common across cancers, and permits cell-surface targeting of specific therapies in preclinical and clinical studies of various solid and circulating tumors. TFR 1 is a member of the TFR family with affinity for transferrin bound to Fe (III). Upon binding, the transferrin-TFR1 complex is internalized via endocytosis after which Fe (III) is dissociated from TF in a pH dependent reaction. As such, TFR plays a critical role in iron uptake by cancer cells and participates in cancer progression and tumor onset.

Further, analysis of published datasets reveals a novel association of increased TFR1 expression with high-risk DLBCL cases. Thus, TFR1 is a valuable pharmacological target. Despite this, targeted delivery of chemotherapy, via TFR, in DLBCL remains poorly understood. Novel, targeted therapeutic approaches for treating DLBCL are needed.

### SUMMARY OF THE DISCLOSURE

This disclosure describes compositions and methods for treating solid tumor cancers including neuroblastoma.

As described below, in a first aspect the present disclosure provides a composition comprising a carbon-nitride dot having a surface comprising carbodiimide cross-linked doxorubicin and transferrin thereupon.

In a second aspect the present disclosure provides a therapeutic composition comprising rituximab, cyclophosphamide, vincristine, prednisone, transferrin, and doxorubicin, wherein the doxorubicin and transferrin is a carbodiimide cross-linked doxorubicin and transferrin on the surface

In one embodiment of the first or second aspect, the nanocarrier contains triazine rings (C3N4) and is synthesized from urea and citric acid.

In one embodiment of the first of second aspect, the nanocarrier contains amine groups, amide groups, and carboxyl groups on its surface and has an excitation wavelength between 450-600 nm.

In one embodiment of the first or second aspect, the composition has a 10-100 fold increased potency against diffuse large B-cell lymphoma in vitro compared to doxorubicin treatment alone and an LD50 to diffuse large B-cell lymphoma is less than 100 nm.

In one embodiment of the second aspect, the carbodiimide cross-linked doxorubicin and transferrin have increased in vivo efficacy.

In a third aspect is a method of treating cancer, in an individual in need thereof, comprising administering the composition containing the nanocarrier to an individual having solid tumor cells overexpressing the transferrin receptor (TFR1).

In one embodiment of the third aspect, the individual has a blood or circulating cancer such as a lymphoma that overexpresses the transferrin receptor (TFR1).

In one embodiment of the third aspect, the nano carrier increases the anti-lymphoma efficacy of doxorubicin on diffuse large B-cell lymphoma cell lines.

In a fourth aspect is a composition comprising, conjugation of a cross-linked doxorubicin and transferrin on the surface of a carbon-nitride dot nanocarrier via a single-chain variable fragment (scFv) against transferrin receptor 1.

In one embodiment of the fourth aspect, the therapeutic composition comprises, rituximab, cyclophosphamide, doxorubicin, vincristine and prednisone, wherein the doxorubicin is cross-linked doxorubicin.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1. TFRC expression in DLBCL patients correlates with poor overall survival. A) Lenz et al. Kaplan-Meier overall survival (OS) analysis of high (red) vs. low (green) TFRC expression in 414 newly diagnosed untreated DLBCL patients. B) Reddy et al. Kaplan-Meier OS analysis of high (red) vs. low (green) TFRC gene expression from 756 DLBCL patients. Dotted lines indicate median OS. Lenz et al. median OS high=4.99 years, low=10.62 years (log-rank high/low HR 1.462, 95% CI 1.041 to 2.053). Reddy et. al median OS high=7.840 years, low=10.40 years (log-rank high/low HR 1.476, 95% CI 1.087 to 2.006).

FIG. 2. Carbon-Nitride Dot Synthesis. A) Synthesis schematic of CNDs with surface functional group illustration. B) The tris-s-triazine structure of CNDs.

FIG. 3. CND-Dox-TF synthesis and validation. A) Schematic of carbon nitride dots-doxorubicin-transferrin (CND-Dox-TF) synthesis. B) Chemical structure of CND-Dox-TF conjugate showing carbodiimide bonds to Dox and holo-TF. C) Normalized photoluminescence emission spectra of CND-Dox-TF excited at specific excitation wavelengths related to each single component (TF-280, CNDs-330-370, Dox-480 nm) which confirms the presence of each individual component in the nanocarrier conjugate.

FIG. 4. Characterization of CND-Dox-TF. A) UV-vis absorption data for the conjugate CND-Dox-TF (black) and all three single components (Dox-red, CNDs-blue, TF-purple). B)-D) Fluorescence emission spectra at maximum excitation wavelength relevant to the absorption for B) TF (excited at 280 nm), C) CNDs (excited at 350-410 nm) and D) Dox (excited at 480 nm). E) Original photoluminescence emission spectra data for CND-Dox-TF for which data in FIG. 2C was normalized off of. F) FTIR spectra for naked CNDs (black) and CND-Dox-TF conjugate (red). G-H) Normalized photoluminescence emission spectra data (G) for CND, CND-Dox-TF, CND-TF, CND-Dox stock compounds stored in −20 Celsius for >1 year and original emission spectra data (H) which was used to produce normalized graphs shown in G).

FIG. 5. CND-Dox-TF has enhanced in vitro cytotoxicity compared to Dox. A) 48-hour viability assays for DLBCL cell lines plated in serial dilutions of Dox and CND-Dox-TF (left) and mean EC50±SEM calculated from three independent 48-hour viability experiments (right). (EC50 500 nM means no significant activity up to that concentration.) B) Time course viability response of BJAB cell line treated with Dox or CND-Dox-TF. Cells were plated in drug for 24 hours then washed out and re-plated in normal media with daily viability assessment. Data normalized to DMSO-treated controls. Statistical analysis reflective of AUC comparing the same dose across treatment groups. Shown are triplicate mean±SEM. C) AnnexinV-PE 7-AAD 24-hour apoptosis assay for BJAB cell line at a range of CND-Dox-TF and Dox doses (left) with percentage of late-apoptotic cells representative of triplicate mean±SEM (right). D) Immunoblot assessment of γ-H2AX (17 kD) for BJAB cells exposed as indicated to Dox or CND-Dox-TF. ****P<0.0001; ***P<0.001 **P<0.01; *P<0.05; ns, nonsignificant (t test). For densitometric analysis, all samples normalized to loading control first followed by normalization to DMSO. ⋅=accurate densitometric evaluation not possible given overly strong signal.

FIG. 6. CND cytotoxicity. A) Mean EC50±SEM calculated from triplicate 48-hour viability experiments. (EC50 500 nM means no activity up to that concentration.) B) Time course viability response of Farage, SU-DHL4 and Riva cell lines treated with Dox and CND-Dox-TF. Cells were plated in drug for 24 hours then washed out and re-plated in normal media with daily viability assessment. Data normalized to DMSO-treated controls. Statistical analysis representative of AUC comparing all timepoints for same dose between treatment groups. Shown are mean triplicate ±SEM. C) AnnexinV-PE 7-AAD 24-hour apoptosis assay for Farage cell line at a range of CND-Dox-TF and Dox doses (left) and percentage of late apoptotic cells representative of mean triplicate ±SEM (right). ****P<0.0001; ***P<0.001; **P<0.01; *P<0.05; ns, nonsignificant (t test).

FIG. 7. CND-Dox-TF mechanism of action. A) Immunoblot analysis of TFR1 (90 kD) expression in DLBCL cell lines with cyclophilin B (24 kD) loading control (top) and BJAB and Farage cells infected and FACS sorted for TFRC overexpression (bottom). B) 48-hour cell viability assays corresponding to cell lines depicted in A plated in serial dilutions of CND-Dox-TF. Data shown are mean quadruplicate ±SEM with P values corresponding to Uninfected vs. TFRC EC50 values. C) 48-hour cell viability assay of BJAB and Farage cell lines plated in serial dilutions of Dox, CND-Dox-TF, CND-TF, and CND-Dox-TF+a constant 250 uM concentration of competitive holo-TF in each well. Data shown are mean quadruplicate ±SEM with P values corresponding to CND-Dox-TF vs. CND-Dox-TF+a constant 250 uM holo-TF EC50 values. D) 48-hour viability response of BJAB and Farage cells treated with dynasore for 48-hours (top panel) and treated with CND-Dox-TF (100 nM)+15 uM or 50 uM dynasore for 48-hours (bottom panel). Shown are mean triplicate ±SEM. E) Fluorescent confocal microscopy images (60× objective) of HEK293 cells incubated for 24 hours with CND-Dox-TF (50 nM), CND (500 nM), CND-Dox (500 nM) or Dox (500 nM). Quantitation of overlap (mean triplicate ±SEM), right panel, of blue nuclear (DAPI) with red fluorescence from both Dox and CNDs. F) Fluorescent confocal microscopy images (63×) of HEK293 cells incubated for 24 hours with a Cyan-TF labeled CND-Dox-TF (50 nM), Doxorubicin (500 nM), and DMSO control. Green fluorescence corresponds to nucleus, blue corresponds to TF and red fluorescence corresponds to inherent signal emitted from Dox+/−CND. ****P<0.0001; ***P<0.001; **P<0.01; *P<0.05; ns, nonsignificant (t test). For densitometric analysis, all samples normalized to loading control first. Samples in A) top panel normalized to highest-expressing cell line BJAB. Samples in A) bottom panel normalized to uninfected basal condition cell line respectively. Scale bar 50 μm.

FIG. 8. Cell surface TFR1 expression and receptor recycling. A) Flow cytometry binding assay of SU-DHL4, BJAB, Riva and Farage cells incubated for 30 minutes with holo-TFCF®568. Shown is mean triplicate ±SEM. B) Flow cytometry histogram plots depicting GFP expression (BL-1 laser) of BJAB and Farage cells before and after FACS sorting for TFRC overexpression. C) 48-hour viability assay for DLBCL cell lines plated in serial dilutions of holo-TF. Shown are mean quadruplicates ±SEM. D) Immunoblot analysis of TFR1 (90 kD) expression in BJAB, Riva, Farage, SU-DHL4 cells treated with 100 nM CND-Dox-TF and CND-TF for up to 24-hours, with β-Actin (42 kD) loading control. For densitometric analysis, all samples normalized to loading control first, followed by normalization to DMSO.

FIG. 9. Rapid nuclear entry by Dox after CND-Dox-TF treatment in DLBCL. Fluorescent confocal microscopy images of BJAB cells incubated for 24 hours with CND-Dox-TF (30 nM), CND (250 nM), CND-Dox (250 nM), and Dox (250 nM). Blue fluorescence (DAPI) corresponds to nucleus while red fluorescence corresponds to inherent signal emitted from both drugs. Images taken at 60× objective. Relative nuclear colocalization (mean triplicate ±SEM) of Dox+/−CND to the right. ***P<0.001; **P<0.01; *P<0.05 (t test).

FIG. 10. HEK293 is a model cell line for elucidating CND-Dox-TF mechanism. A) 24-hour viability response of HEK293 cells plated in serial dilution of Dox, CND-Dox-TF, CND-Dox, and CND. B) 24-hour viability response of HEK293 cells plated in serial dilution of Cyan-tagged TF CND-Dox-TF (CND-Dox-CFPTF) and CND-Dox-TF. Shown are mean quadruplicates ±SEM.

FIG. 11. CND-Dox-TF working dose identified. A) Body weight by nadir (day 8) of 3 non-tumor bearing NSG mice per group, treated with a range of Dox and CND conjugate doses. B) Organ H&E pathology from control (untreated) NSG mouse and mice treated with CND-Dox-TF at both dosing levels. Scale bar 50 μm (40× objective for all).

FIG. 12. CND-Dox-TF has improved toxicity profile compared to Dox. A) Fresh-frozen paraffin-embedded tumor from DLBCL PDX model stained for TFR1 (CD71) expression. B)-D): Four groups of 10 NSG mice were implanted with DLBCL PDX Tumor (S5) and treated with MTD Dox (3.3 mg/kg), WD CND-Dox-TF (33.0 mg/kg) and CND-Dox(*), CND(*) on day 0, 14, and 24. Predetermined survival endpoints were tumor volume >1500 mm3, continuous weight loss >20%, or other signs of morbidity. B) Overall survival of all treatment groups. CD) Tumor volume measured ×2 weekly via ultrasound. D) Body weight measured daily until predetermined endpoint reached. *CND-Dox and CND are molar equivalents to MTD CND-Dox-TF. ****P<0.0001; NS, nonsignificant (t test). Scale bar 50 μm (40× objective).

FIG. 13. R-nanoCHOP improves overall survival compared to R-CHOP in DLBCL PDX-bearing NSG mice. 22 NSG mice were implanted with DLBCL PDX tumor (S8B) and randomized to two groups. After death of one animal prior to engraftment, remaining animals were treated with R-CHOP (n=11) or R-nanoCHOP (n=10) once on day 1 of every 21 days. Predetermined survival endpoints were tumor volume >1500 mm3, weight loss >20%, or other signs of morbidity. A) Average tumor volume ±SEM measured twice weekly via ultrasound. B) Average daily body weight ±SEM of surviving animals. C) Overall survival of all treatment groups. ****P<0.0001 (Mantel-Cox). A-C are mean±SEM of at least ten technical replicates representative of one independent experiment.

FIG. 14. R-nanoCHOP treatment has favorable toxicity profile in non-malignant organs. A) Tumor volume representative of each individual mouse per treatment group. B) Body weight representative of each individual mouse per treatment group. C) Kidney and Lung H&E pathology collected from R-CHOP and R-nanoCHOP treated mice at predetermined survival endpoints. Scale bar 50 μm (20× objective for all).

FIG. 15. R-nanoCHOP treatment has favorable toxicity profile. A) Heart, Colon, Small Intestine, Bone Marrow, Spleen, Liver H&E pathology collected from R-CHOP and R-nanoCHOP treated mice at predetermined survival endpoints. B) Immunoblot analysis of TFR1 (90 kD) expression in HEK293, BJAB, 3T3 and A20 cell lines. C) 48-hour viability assays for A20 cells plated in serial dilution of Dox and CND-Dox-TF. D) Flow cytometry binding assay of A20 and BJAB cells incubated for 30 minutes with DMSO, holo-TFCF®568, or CND-Dox-TF. Scale bar 50 μm (20× objective for all). For densitometric analysis, all samples normalized to loading control first followed by normalization to respective lower TFR1-expressing cell lines.

FIG. 16. Characterization of NanoDox-sc. A) Schematic of CND-Dox conjugated with anti-TFR 1 scFv (left) and successful characterization of all substrates attached (right). B) Relative EC50 values calculated from triplicate viability experiments. Mean±SEM. ****P<0.0001; ***P<0.001 **P<0.01; *P<0.05; NS=not significant (t-test). C) Dox quantification with circular dichroism against a standard Dox series depicting a 1:2 CND:Dox ratio in CND-Dox-scFvTF and 1:18 CND:Dox ratio in CND-Dox-TF.

### DETAILED DESCRIPTION OF THE PREFERRED EMBODIMENTS

Provided herein are therapeutic compositions for treatment of circulating cancer, specifically DLBCL. Also provided herein are methods of treating cancer using a carbon-nitride dot nanocarriers.

It is to be understood that the particular aspects of the specification as described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

Unless otherwise clear from context, all numerical values provided herein can be modified by the term about. Unless specifically stated or obvious from context, as used herein, the term “about” is understood as within a range of normal tolerance in the art, for example, within two standard deviations of the mean. About also includes the exact amount. For example, “about 5%” means “about 5%” and also “5%.” The term “about” can also refer to ±10% of a given value or range of values. About can be understood as within 10%, 9%, 8%, 7%, 6%, 5%, 4%, 3%, 2%, 1%, 0.5%, 0.1 (Yo, 0.05%, or 0.01% of the stated value. Therefore, about 5% also means 4.5%-5.5%, for example.

One of ordinary skill in the art, will understand that values herein that are expressed as ranges can assume any specific value or sub-range within the stated ranges in different embodiments of the disclosure, to the tenth of the unit of the lower limit of the range, unless the context clearly dictates otherwise. As such, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “such as” means, and is used interchangeably with, the phrase “such as, for example” or “such as but not limited.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to an amount of a therapeutic agent, such as a CDT composition, which when administered to a subject, is sufficient to effect treatment for a disease or disorder described herein, such as reducing survival or spread of cancer cells and tumors. The amount of a composition which constitutes a “therapeutically effective amount” or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of one or more symptoms of the disease or disorder.

“Subject” refers to a warm-blooded animal such as a mammal, preferably a human, which is afflicted with, or has the potential to be afflicted with one or more diseases and disorders described herein.

“Pharmaceutical composition” as used herein refers to a composition that includes one or more therapeutic agents disclosed herein, such as CDT compositions, a pharmaceutically acceptable carrier, a solvent, an adjuvant, and/or a diluent, or any combination thereof.

In view of the present disclosure, the methods and compositions described herein can be configured by the person of ordinary skill in the art to meet the desired need.

### Diffuse Lame B-Cell Lymphoma (DLBCL)

DLBCL comprises a third of non-Hodgkin lymphoma (NHL) in the United States, making it the most common hematologic malignancy (1). Frontline R-CHOP (rituximab, cyclophosphamide, doxorubicin, vincristine and prednisone) is effective in ˜60%, but patients with relapsed or refractory (rel/ref) disease following frontline therapy have poor prognosis, with only about 1 in 10 achieving long-term disease-free survival, typically requiring salvage chemoimmunotherapy followed by bone marrow transplantation (2). Overall, there is substantial unmet need in DLBCL, with at least one in three diagnosed patients ultimately dying.

The anthracycline chemotherapeutic doxorubicin (Dox) remains the most active drug against DLBCL, serving as the backbone of R-CHOP and most other standard frontline combination treatment regimens, more than five decades after the compound's introduction (3). Clinical use of Dox is limited by toxicities to bone marrow and cardiomyocytes, especially in patients with prior anthracycline exposure, resulting in lifetime cumulative and dose-dependent cardiotoxicity (4-6).

R-CHOP (rituximab, cyclophosphamide, doxorubicin, vincristine, prednisone), for example, cures diffuse large B-cell Lymphoma (DLBCL), the most common lymphoid malignancy in the United States, greater than 60% of the time. Patients with relapsed/refractory disease, however, have poor prognosis and require new options. Advances in nanotechnology provide new opportunities to widen therapeutic windows for existing drugs by enhancing delivery to tumor cells and limiting toxicities to non-malignant tissues. Carbon-Nitride Dots (CND) are novel nanocarriers we have developed that can be conjugated with a diverse range of molecules and have an established safe pharmacologic profile. Here, we sought CND-based enhancement of Dox's anti-lymphoma activities. Targeted delivery of Dox could alleviate unwanted effects by sparing non-malignant tissues while maintaining antitumor efficacy.

### Transferrin Receptor 1 (TFR1)

The transferrin receptor 1 (TFR1), also known as CD71, is a ubiquitous cell-surface receptor found at low levels in normal human tissue, serving as the point of entry for iron bound to its ligand transferrin (TF) (7). TF carrying two atoms of Fe3+ (holo-TF) undergoes clathrin-mediated endocytosis upon TFR1 binding, followed by Fe reduction and release to fuel metabolism and proliferative pathways. Tumors often meet high iron demands through TFR1 overexpression (8). TFR1 is expressed at higher levels in a variety of cancers, a well-established potential therapeutic window for targeted therapeutic delivery (9-18). Preclinical studies have exploited this in breast cancer (19-23), glioma, and melanoma (24-26).

### Carbon Dots and Carbon Nitride Dots

Carbon dots (CDs) are low-cost photoluminescent nanoparticles with a gaussian size distribution of 2-8 nm with varying mean diameters dependent on syntheses techniques (27). CDs have reduced toxicities and environmental hazards compared to first-generation quantum dots synthesized from semiconductor metals (28-30). Prior work demonstrates utility of CDs as imaging reagents through incorporation of photoluminescent moieties (31-34). Intravenous (i.v.) dosing results in homogeneous distribution of CDs to different organs including the bladder, kidney, liver, spleen, brain, and heart, followed by rapid excretion in urine (35-38). Third-generation nanoparticles called carbon nitride-dots (CNDs) which have a gaussian size distribution of 1-3.8 nm with a mean diameter of 2.4 nm, formed from C3N4 triazine polymers (39). CNDs have excellent properties as potential therapeutic scaffolds, including enhanced excitation-dependent photoluminescence, reduced size, and improved stability compared to CDs (40).

### Compositions

In one aspect of the disclosure pharmaceutical compositions contemplated herein include a composition comprising comprising carbodiimide cross-linked doxorubicin and transferrin on the surface of a carbon-nitride dot nanocarrier.

Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In another embodiment, the nanocarrier can contain triazine rings (C₃N₄) and is synthesized from urea and citric acid.

Non-limiting examples of therapeutic compositions contemplated for use in the present disclosure include a carbodiimide cross-linked doxorubicin and transferrin on the surface of a carbon-nitride dot nanocarrier. In some embodiments the composition is comprised of rituximab, cyclophosphamide, doxorubicin, vincristine and prednisone (R-CHOP) wherein doxorubicin is replaced with a carbodiimide cross-linked doxorubicin and transferrin on the surface of a carbon-nitride dot nanocarrier (R-nanoCHOP). Additional therapeutic compositions include chemotherapy drugs.

In one embodiment the therapeutic composition contains, but is not limited to, amine groups, amide groups, hydroxyl groups and carboxyl groups on its surface. In yet another embodiments the nanocarrier has an excitation wavelength from about 450 nm to about 600 nm. In some aspects the excitation is wavelength is about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, or about 650 nanometers.

In yet another embodiment the therapeutic target has about a 5 to about 120 fold, about 10 to 100 fold, or about 25 to 50 fold increased potency against diffuse large B-cell lymphoma in vitro compared to doxorubicin treatment alone. In some embodiments the increased potency is about 10, about 15, about 20, about 25, about 30, about 35, about 40, about 45, about 50, about 55, about 60, about 65, about 70, about 75, about 80, about 85, about 90, about 95, or about 100 fold increased potency.

In some embodiments the LD₅₀ to diffuse large B-cell lymphoma is less than 120 nm or less than 100 nm. In some embodiments the LD₅₀ to diffuse large B-cell lymphoma is about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, or about 120 nm.

In a further embodiment the carbodiimide cross-linked doxorubicin and transferrin have increases in vivo efficacy when compared to controls.

Carbon nitride dots-doxorubicin-transferrin (CDT) compositions of the present disclosure can be administered through a variety of routes and in various compositions. For example, compositions containing CDTs can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, the CDT is administered orally, and the subject is dosed on a daily basis.

The therapeutic agents described herein (e.g., CDT), or compositions thereof, will generally be used in an amount effective to achieve the intended result, for example, in an amount effective to provide a therapeutic benefit to subject having the particular disease being treated. As used herein, “therapeutic benefit” refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Non-limiting examples of contemplated secondary therapeutic agents include, but is not limited to gemcitabine, Rituximab, Cyclophosphamide, Doxorubicin, Vincristine, Prednisone 0.2 mg/kg, and a gramental variable (ScFv) of transferrin.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of compositions containing CDT disclosed herein and secondary therapeutic agents can be in the range of from about 0.0001 mg/kg/day to about 100 mg/kg/day, or about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition having a concentration of a CDT of at about 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 μM in a pharmaceutically acceptable carrier.

In some embodiments, methods of treating cancer, such as diffuse large B-cell lymphoma (DLBCL) expressing the TFR1, in a subject in need thereof include administering to the subject a therapeutically effective amount of a composition containing CDT herein and optionally a second therapy and/or secondary therapeutic agent. Contemplated treatable cancers can include DLBCL at various stages (e.g., stage I, II, or III cancers) or as diagnosed using the International Prognostic Index (IPI). In another embodiment the compositions can be used to treat solid, blood, or circulating cancers expressing TFR1. Such cancers include, but are not limited to cancers of the breast, prostate, lung, pancreatic, liver, lymph leukemia, brain, and head and neck, as well as lymphoma, and myeloma.

In some embodiments, the therapeutic methods contemplated herein include administering to the subject a pharmaceutical composition to the subject orally and/or intravenously.

In some embodiments, the therapeutic methods contemplated herein include administering to the subject a pharmaceutical composition including CDT or R-nanoCHOP and one or more secondary therapeutic agents. In other embodiments, the therapeutic methods include administering a first pharmaceutical composition including a CDT and a second pharmaceutical composition including one or more secondary therapeutic agents.

Having described the invention in detail and by reference to specific aspects and/or embodiments thereof, it will be apparent that modifications and variations are possible without departing from the scope of the invention defined in the appended claims. More specifically, although some aspects of the present invention can be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

The invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

### EXAMPLES

### Overview

Disclosed herein is novel nanocarrier delivery of chemotherapy via TFR1-mediated endocytosis, assessing this target for the first time in DLBCL. Doxorubicin (Dox) and transferrin (TF) were cross-linked to CNDs as set forth herein. In vitro, CND-Dox-TF (CDT) was 10-100 times more potent than Dox alone against DLBCL cell lines. Gain- and loss-of-function studies and fluorescent confocal microscopy confirmed dependence of these effects on TFR1-mediated endocytosis. In contrast to previous therapeutics directly linking Dox and TF, cytotoxicity of CDT resulted from nuclear entry by Dox promoting double-stranded DNA breaks and apoptosis. CDT proved safe to administer in vivo, and when incorporated into standard frontline chemoimmunotherapy in place of Dox, it improved overall survival (OS) by controlling patient derived xenograft (PDX) tumors with greatly reduced host toxicities. Nanocarrier-mediated Dox delivery to cell-surface TFR1 therefore warrants optimization as a potential new therapeutic option in DLBCL.

### Methods

### Carbon-Nitride Dot Synthesis

Anhydrous citric acid (BDH) was obtained from VWR (West Chester, Pa.). Urea was acquired from Eastman Kodak Company (NY, USA). Doxorubicin hydrochloride and holo-transferrin (human plasma) were from TCI America Inc. (OR, USA) and EMD Millipore Corp. (MA, USA), respectively. N-Hydroxysuccinimide (NHS) and 1-ethyl-3-(3-dimethylaminopropyl) carbodiimide (EDC) were purchased from Millipore-Sigma (St. Louis, Mo.). 3500 Da molecular weight cut-off dialysis tubing was from Thermo-Scientific (Rockford, Ill.) while the 100-500 Da molecular weight cut-off tubing were bought from Spectrum Labs Inc., (CA, USA). The deionized (DI) water used was ultrapure (type I) water purified using a Millipore Direct-Q 3 water purification system acquired from EMD Millipore Corp. with a surface tension of 72.6 mN·m-1, a resistivity of 18 MΩ·cm and a pH of 6.6±0.3 at 20.0±0.5° C.

The synthesis of carbon-nitride dots (CNDs) was performed using a simple hydrothermal microwave process using citric acid and urea as reported previously (39). A summary of this synthesis involves a 0.5 g of each citric acid and urea dissolving in 25 mL of deionized water for overnight vigorous stirring before a microwave thermal treatment for 7 min under 700 W. The resultant solid residue was sonicated in 20 mL water and centrifuged for 30 min twice to remove large particles from the CND dispersion. Filter membranes (0.2 μm) were used to filter the dispersion and filtrate was dialyzed in a 100-500 Da dialysis tubing for 5 days against 4 L DI water with regular water changes every 24 h. The water dispersion was evaporated to obtain the solid CNDs product. The characterization of the CNDs was performed and reproducibility confirmed as reported (39).

### Synthesis and Characterization of Carbon-Nitride Dot-Doxorubicin-Transferrin (CND-Dox-TF)

The as-synthesized CNDs were used for the preparation of the conjugate. CNDs (8 mg) were first dissolved in 3 mL of phosphate buffered saline (PBS, pH 7.4 at 25 mM) and were mixed with EDC (17 mg in 1 ml PBS) before stirring at room temperature for 30 min. Then, NHS (10.2 mg in 1 mL PBS) was added to the above mixture and left for stirring for another 30 min. Then 6 mg of doxorubicin hydrochloride (Dox) was dissolved in 0.5:0.5 mL DMSO: PBS, added to the reaction mixture and stirred for 30 min, before the addition of holo-transferrin (TF, 3 mg in 1 mL PBS). The reaction was stirred overnight and transferred into a 3.5 kDa dialysis tubing for dialysis against 2 L Distilled (DI) water for 4 days with water changes every 24 hours, (40, 41). The resultant dialyzed solution was freeze-dried to yield the lyophilized product.

The as-prepared CND-Dox-TF conjugate was subjected to different characterization techniques to confirm the existence of the said conjugate compound. UV-Vis absorption characterization was performed using a Cary 100 UV-Vis spectrophotometer (Agilent Technologies) in aqueous medium in a 1 cm quartz cuvette (Starna Cells). For the luminescent emission observations, a Horiba Jobin Yvon Fluorolog-3 spectrometer was used (in 1 cm path length quartz cuvette) using a slit width of 5 nm for both excitation and emission. OriginPro 9.1 was used to create the normalization of the emission spectra with the y-axis normalized to 1. Fourier transform infrared (FTIR) spectra was recorded with a universal ATR sampling accessory (Perkin-Elmer Frontier) using air as the background. Samples were also analyzed through mass spectroscopy using matrix-assisted laser desorption ionization time of flight (MALDI-TOF) (Bruker).

### Prognostic Correlation

Overall survival analysis based on TFRC expression for previously untreated DLBCL patients was performed using the SurvExpress online tool (42) for both the Lenz (GEO ID #GSE10846) (43) and Reddy (European Genome-phenome Archive at the European Bioinformatics Institute (EGAS00001002606) (44), datasets. Analysis for both datasets was conducted using the Maximize Risk Groups function in the SurvExpress online tool (42).

### Cell Culture

All cells lines were verified by STR fingerprinting and assessed for mycoplasma contamination. Culture media for SU-DHL4, BJAB, and Riva (DSMZ); Farage and Toledo (ATCC); HBL1 and Karpas-422); and A20 were RPMI 1640 supplemented with 10% fetal bovine serum (FBS), Penicillin/Streptomycin (P/S), and mycoplasma inhibitor plasmocin prophylactic (P/P) (ant-mpp). OCI-Ly19 (ATCC) was cultured in IMEM supplemented with 20% FBS, P/S and P/P. HEK293 and 3t3 (ATCC) was cultured in DMEM supplemented with 10% FBS, P/S, and P/P.

### Cell Viability

For 24-48 hr assessments, cells were seeded at 5000 cells per well in a 96-well plate under serial drug dilutions. For delayed drug effect viability, cells were seeded at 500,000 cells per well in a 6-well plate on day 0 and treated with drug for 24 hrs, after which cells were washed ×2 and plated in normal cell media without drug. Viability was assessed using Cell Titer Glo (Promega #G7573) according to manufacturer's protocol. Luminescence was measured using BioTek Synergy HT plate reader. EC50s were calculated using nonlinear fit regression analysis in GraphPad Prism 8. Apoptosis assessment was conducted with BD Biosciences reagent (#559763) using Attune NxT flow cytometer.

### Antibodies

Antibodies used in experiments herein include CD71 (Cell Signaling Technology; #13113S; or Thermofisher #MA532500), Phospho-Histone H2A.X (Cell Signaling Technology #9718S), β-Actin (Cell Signaling Technology #4970S), and Cyclophilin B (#PA1-027A).

### Protein Extraction, Quantification, and Immunoblotting

Cells were seeded at 500,000/mL and incubated as indicated. Proteins were extracted using RIPA (VWR, Radnor, Pa., USA), Phosphatase Halt (Thermo #78428). Proteins were quantified using the BCA assay (Thermofisher Scientific, Waltham, Mass., USA) with 20 ug loaded per lane for Western blotting. All blots were developed using autoradiography film (VWR, Radnor, Pa., USA) or Li-Cor Odyssey Fc imaging system after incubation with antibodies indicated above. Densitometric analysis conducted using Li-Cor affiliated ImageStudio software, with all analyses normalized to loading controls. All antibodies were used per the manufacturer recommended dilutions.

### Microscopy

Cells were seeded at 50,000 cells per well in a 12-well plate and treated with either vehicle or drug. BJAB cells were fixed with 4% paraformaldehyde and permeabilized with 1% NP40 followed by staining for DAPI (Thermofisher Scientific, Waltham, Mass., USA). Cells were then imaged (60×) using a Leica DM4 B microscope. HEK293 cells had the nucleus stained for DAPI (Thermo #R37605) or GFP (Thermo #C10602) per the manufacturer protocol. Cells were live-imaged (63× objective) using Leica Sp5 confocal microscop and images collected and analyzed using ImageJ software.

### TFR1 Overexpression

Human TFRC cDNA (HsCD00044911) was purchased from the DNASU Plasmid Repository (Tempe, Ariz., USA) and was subsequently cloned into pLVX-IRES-ZsGreenl vector (Clontech Laboratories, Mountain View, Calif., USA). Recombinant plasmids, together with packaging/envelope plasmids psPAX2 and pMD2.G (Addgene, Cambridge, Mass., USA), were co-transfected into HEK293 cells using Lipofectamine 3000 (Invitrogen, Carlsbad, Calif., USA) following manufacturer's instructions. Cell media was changed at 24 hours after transfection, and viral particles collected at 48 hours and 72 hours post transfection.

For viral transduction, BJAB and Farage cells were infected with harvested virus by spinoculation. Briefly, the cells were spun at 1800 rpm for 45 mins at room temperature. Cells were infected twice per day for a total of 4 infections. Fresh media was replenished the day after infections and cells were expanded. BD FACSAria II cell sorter was used to sort GFP positive cells. Cells transduced with empty vector were used as negative controls.

### Binding Assay

Briefly, 500,000 cells were centrifuged at 800 g×5 minutes, washed twice with cold PBS, and moved to ice. Cells were incubated with holo-TFCF®568 (25 ug/mL) and CDT (500 nM) for 30 minutes on ice, followed by two washes with cold PBS, and then measured using an Attune NxT flow cytometer using a YL1 laser.

### Inhibitors

Holo-Transferrin (616397-500MG-M) was purchased from Millipore Sigma. Fluorescently conjugated Holo-Transferrin (CF®405S and CF®568) was purchased from Biotium. Dynasore (S8047) was purchased from Selleckchem. Rituximab, Cyclophosphamide, Doxorubicin, Vincristine and Prednisone were provided by the Sylvester Comprehensive Cancer Center chemotherapy pharmacy.

### In Vivo Studies

All animal studies were performed under the approval of the University of Miami Institutional Animal Care and Use committee. All mice in this study were NOD scid gamma (NSG) males >8 weeks of age. For tumor-bearing experiments, DLBCL PDX DFBL-75549 tumor model was obtained and engrafted mice through surgical dorsal tumor implantation. Tumor volume (TV) was measured by ultrasound (Vevo 3100, Visualsonics) with a predetermined survival endpoint of TV>1500 mm3. A continuous body weight loss of >20% was also a predetermined survival endpoint. For dose-finding experiments, mice were dosed intravenously on day 0, day 14, and day 24 and observed for changes in body weight. For R-nanoCHOP vs. R-CHOP tumor-bearing experiments, mice were dosed with all drugs intravenously once on day 1 of every 21 days, with exception to Prednisone administered orally. The following drug doses were used: Rituximab 20 mg/kg, Cyclophosphamide 40 mg/kg, Doxorubicin 3.3 mg/kg, CDT 33 mg/kg, Vincristine 0.5 mg/kg, Prednisone 0.2 mg/kg.

Formalin-fixed paraffin-embedded tissue sections, produced per standard protocols, were used to make H&E-stained and immunohistochemistry (IHC) pathology slides. Antibodies were used as per above, when applicable.

### Statistical Analysis

Two tailed Student t test was carried out for all data using the GraphPad t test calculator, with P<0.05 considered statistically significant with a 95% confidence interval. Area under the curve (AUC) carried out for delayed onset toxicity assessments using the GraphPad AUC function with subsequent Student t test carried out based off total area, SEM and n values, with P<0.05 considered statistically significant with a 95% confidence interval. All experiments reported are the mean triplicate or quadruplicate+SEM of three independent replicates unless otherwise stated in the figure legend. OS analysis employed log-rank (Mantel-Cox) statistics in Prism 8 software, with p<0.05 considered significant.

### Results

### Example 1: High TFRC in DLBCL Correlates with Inferior Outcome after Frontline Therapy

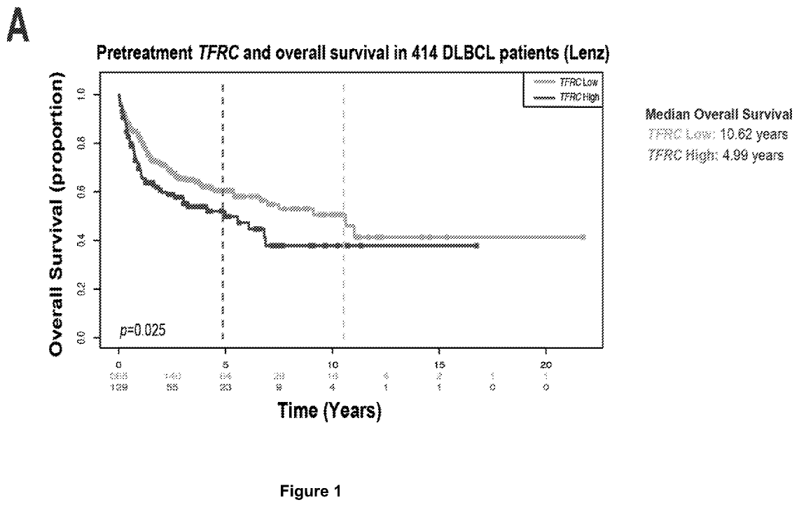

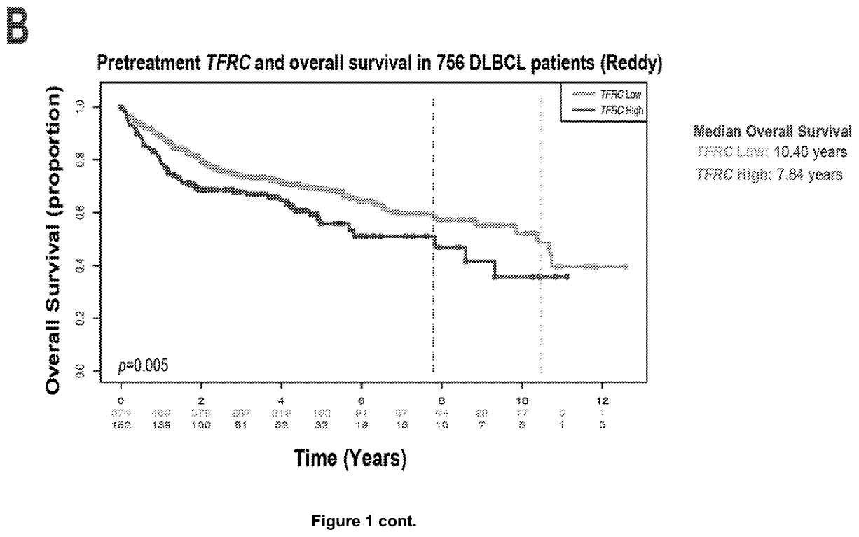

TFR1 expression correlates with worse clinical outcomes in solid tumor malignancies (14-18). Though work nearly 40 years ago suggested worse prognosis in aggressive lymphomas with high TFR1 expression, this has not been analyzed in DLBCL as currently defined and treated. Expression of TFRC, the gene encoding TFR1, was examined in two independent published DLBCL gene-expression datasets. Analysis of chip-based gene-expression data from Lenz and colleagues on 414 previously untreated DLBCL tumors showed significantly worse overall survival (OS) after frontline therapy for patients with high TFRC (p=0.025, HR 1.44 (95% CI 1.05-1.97), FIG. 1A) (42, 43). All these patients were treated with either R-CHOP (233) or CHOP (181). More recently, Reddy et al. performed RNA-seq on 756 newly diagnosed DLBCL cases from patients uniformly treated with rituximab-containing standard frontline combination therapy. High TFRC expression again carried significantly worse OS in these data (p=0.005, HR 1.48 (95% CI 1.12-1.95), FIG. 1B) (44). A known marker of highly proliferative cells, high TFRC identifies DLBCL cases, under current diagnostic criteria, at high risk to be failed by R-CHOP and other standard frontline treatments.

### Example 2: Generation of CND-Dox-TF

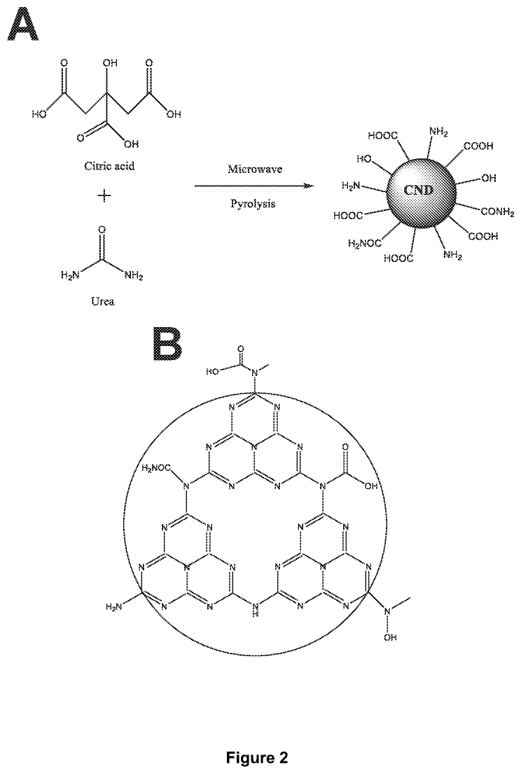

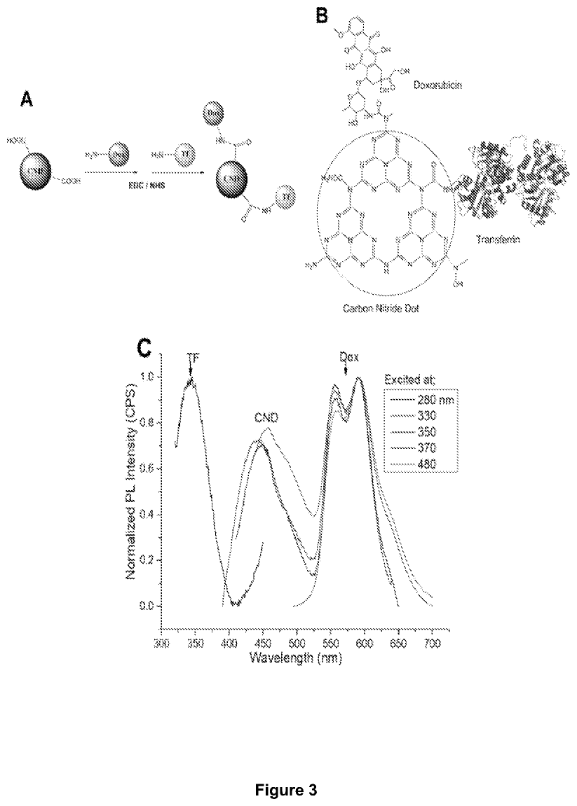

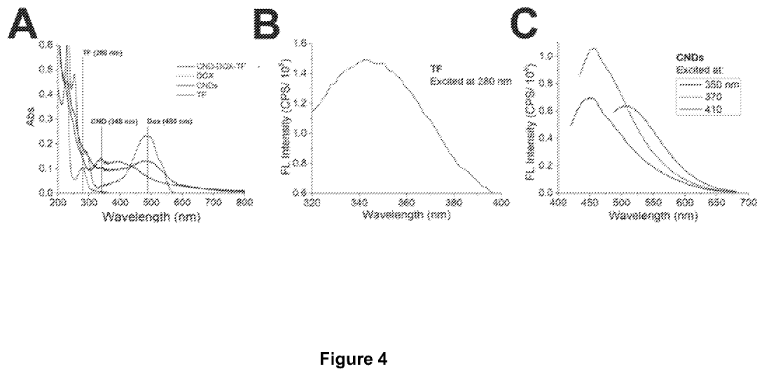

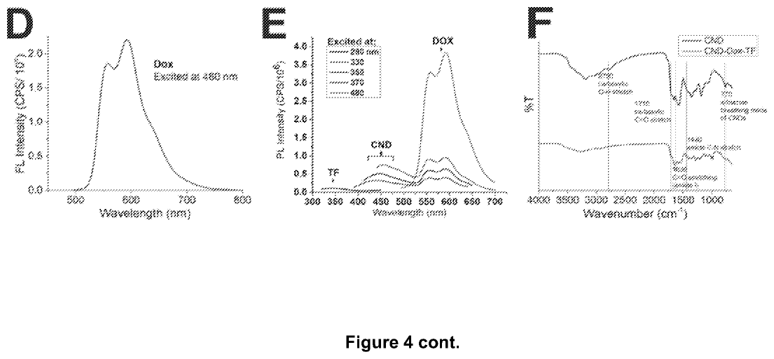

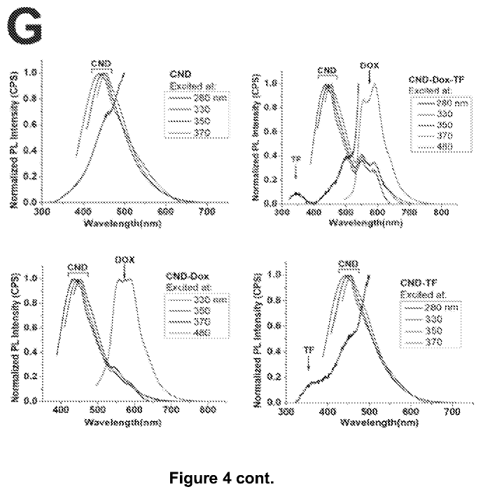

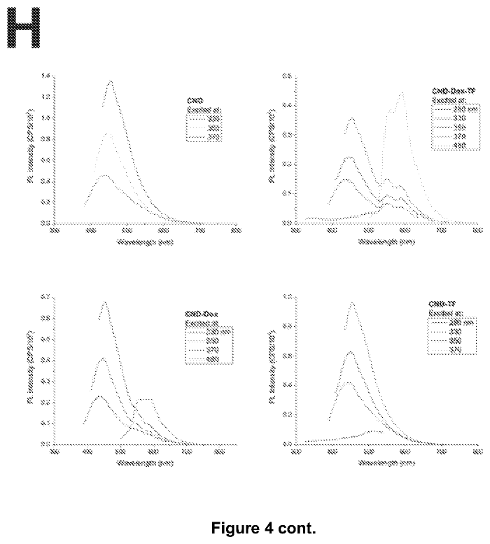

CNDs from urea and citric acid were synthesized using a previously published hydrothermal microwave technique (39). The resulting CNDs were confirmed to consist of a tris-s-triazine structure containing C and N, with high abundance of amine, amide, and carboxylic functional groups (FIG. 2). 1-ethyl-3-(3-dimethylaminopropyl (EDC)/N-Hydroxysuccinimide (NHS) bioconjugation was used to form carbodiimide bonds between CND carboxylic (COOH) groups and amino (NH2) groups on Dox and holo-TF, forming stable covalent bonds, yielding the CDT chemotherapeutic (FIG. 3A-3B) (40). Ultraviolet (UV) absorption and photoluminescent spectra analyses of the individual CDT components was used to confirm the presence of each in CDT (FIGS. 3C and 4A-4E). Fourier-transform infrared (FTIR) spectra analysis comparing unconjugated CNDs vs. CDT confirmed structural bond presence of Dox and TF in CDT (FIG. 4F). To assess long-term compound stability, photoluminescent spectra analyses was performed on stock unconjugated and conjugated CND compounds stored at −20 Celsius conditions for >1 year, confirming the stable conjugation of each component (FIGS. 4G-H). Collectively these results illustrate successful synthesis and stability of CND compounds and the novel CDT chemotherapeutic nanocarrier designed for targeted delivery of Dox to TFR1-expressing cells.

### Example 3: CDT is Exponentially More Potent than Dox Against DLBCL Cell Lines

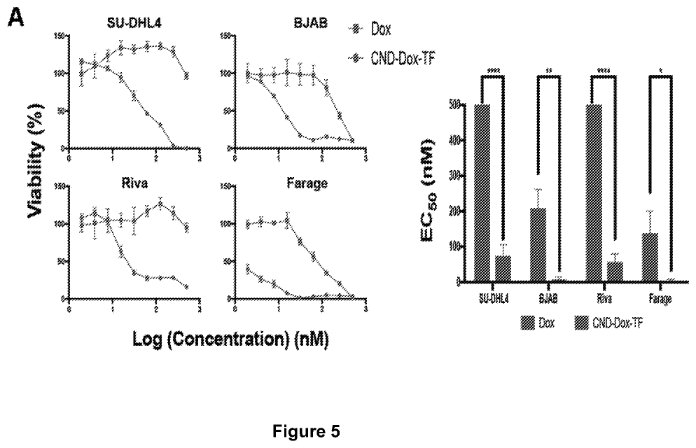

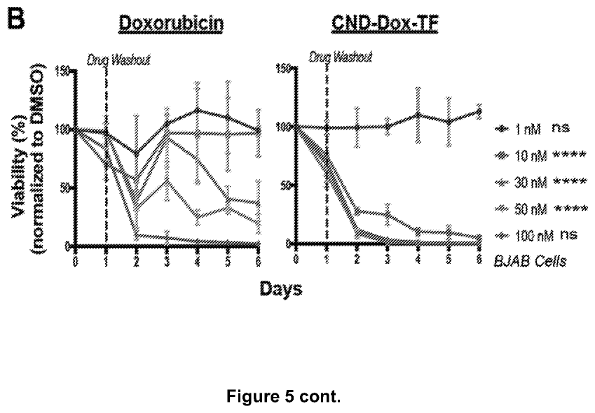

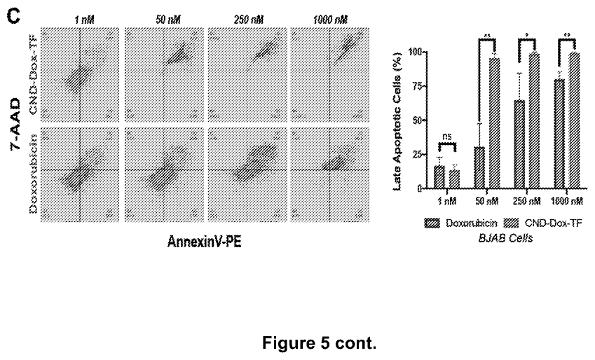

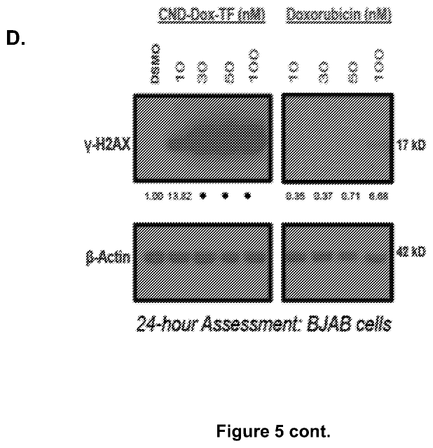

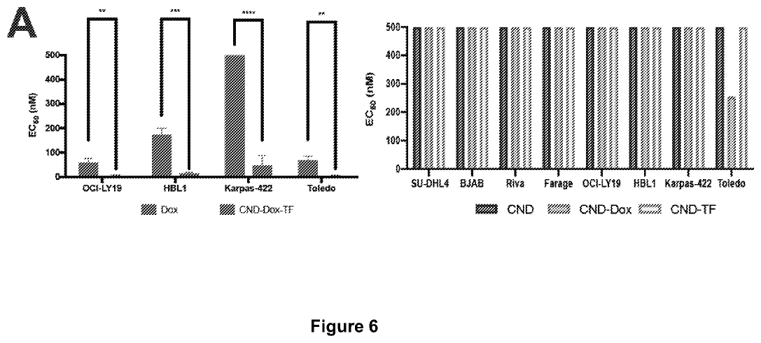

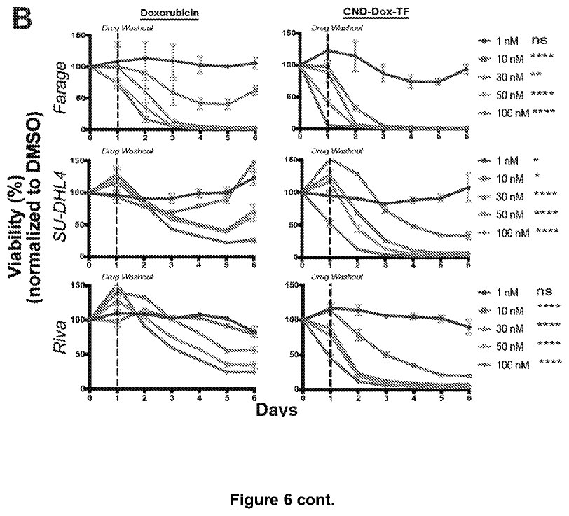

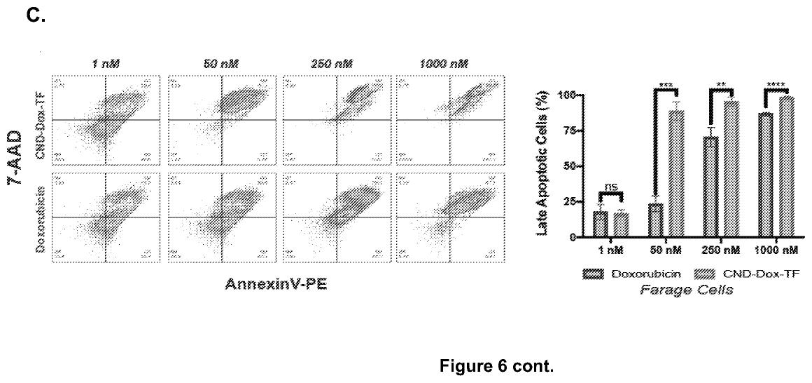

The activity of CDT was compared to single-agent Dox in vitro. DLBCL lines SU-DHL-4, BJAB, Riva, Farage, OCI-Ly19, HBL1, Karpas-422, and Toledo were dramatically more sensitive to CDT (CND-Dox-TF) than molar-equivalent Dox (FIGS. 5A and 6A). Unconjugated (CND) and single-conjugate CND compounds (CND-Dox and CND-TF) had weak or no negative effects on cell viability (FIG. 6A), suggesting CDT activity depends on dual conjugation of Dox and TF to CNDs. Dox may have several different cellular effects, with nuclear entry and DNA damage considered most important against tumors (45). Additionally, onset of apoptosis from Dox DNA damage may be delayed beyond its terminal half-life (46). BJAB, Farage, SU-DHL4, and Riva cells were treated with CDT or Dox for 24 hours at a range of doses, followed by drug washout and continuous cell viability tracking over 6 days. Strikingly, 10 nM CDT induced rapid cytotoxicity, with complete loss of cell viability that never recovered over the time-course, an effect seen only with much higher doses of Dox (FIGS. 5B and 6B). Flow cytometry confirmed entry into apoptosis in CDT-treated BJAB and Farage cells at significantly lower doses compared to Dox (FIGS. 5C and 6C). Western blot analysis of γ-H2AX, the classic DNA-damage marker, further confirmed rapid onset of double-stranded DNA breaks by CDT at dramatically lower doses than Dox in BJAB cells (FIG. 5D). CDT therefore is exponentially more potent than Dox against DLBCL cells, inducing rapid DNA damage and onset of apoptosis.

### Example 4: CDT Promotes Rapid Nuclear Entry by Dox after TFR1-Mediated Endocytosis

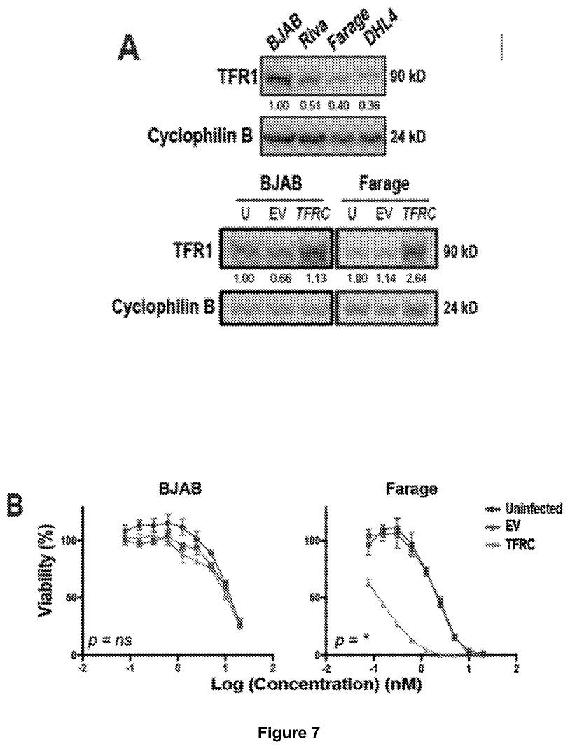

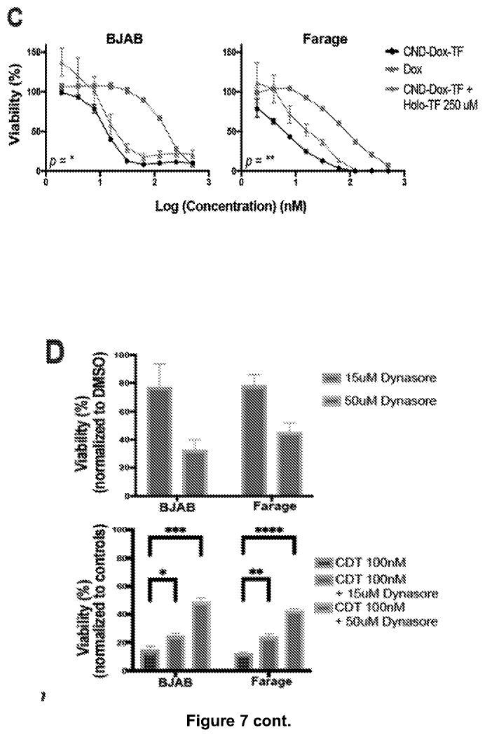

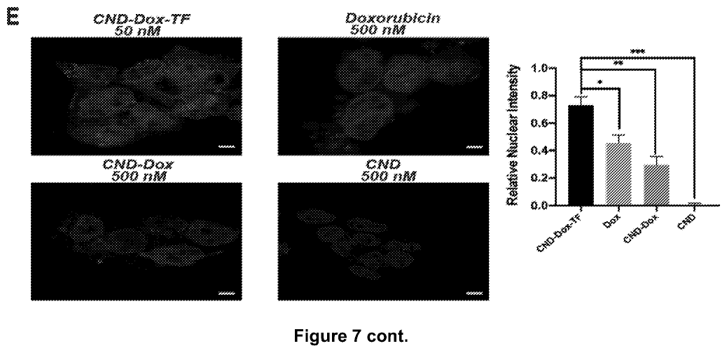

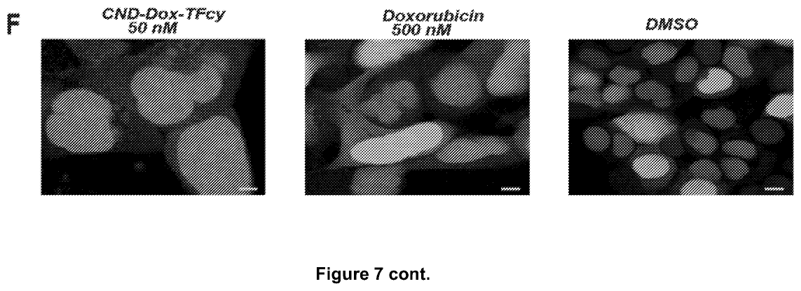

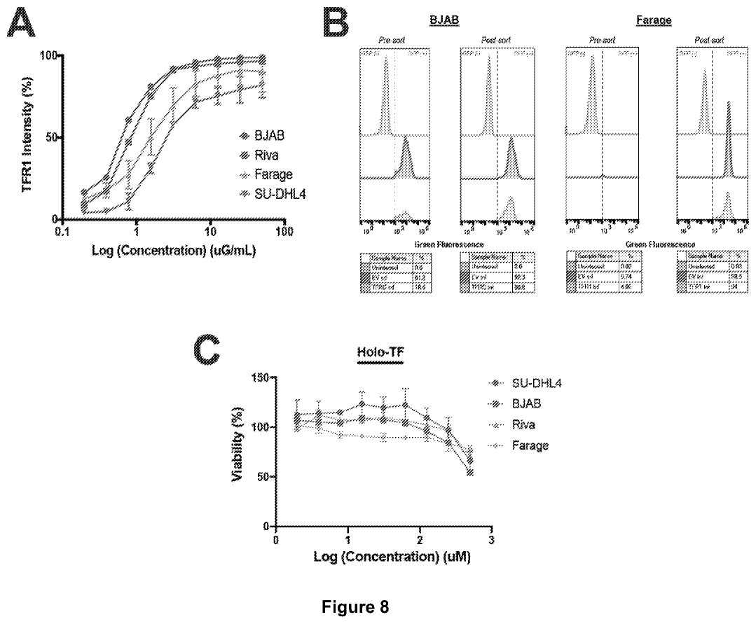

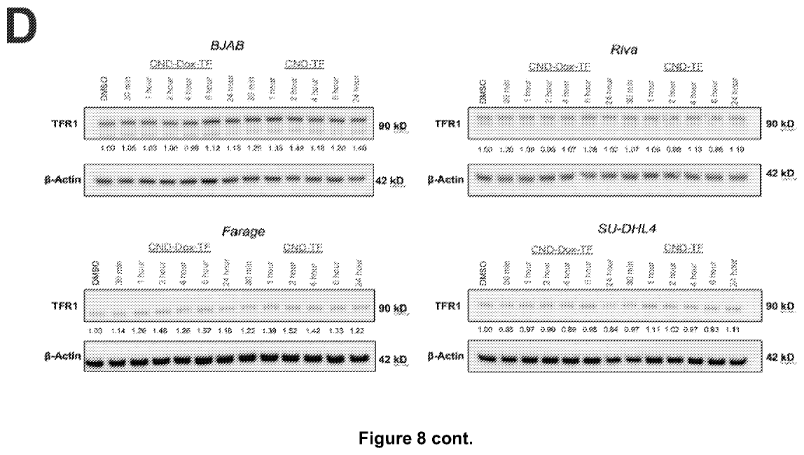

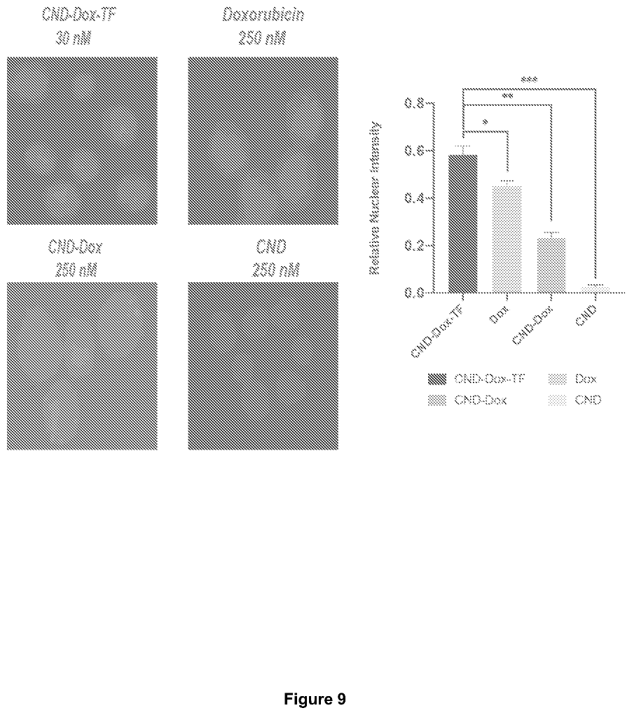

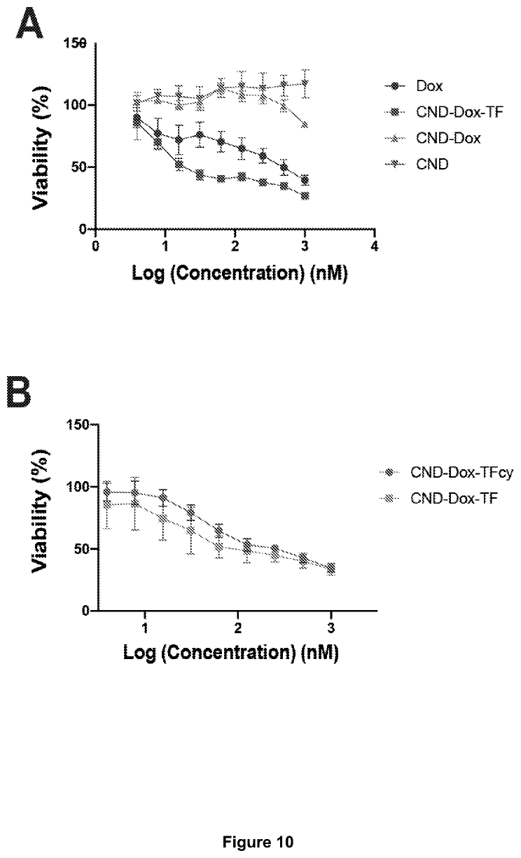

To investigate whether CDT activity was due to TF binding to cell-surface TFR1, TFRC was overexpressed in high-TFR1 expressing BJAB and low-TFR1 expressing Farage cell lines (FIGS. 7A, 8A, 8B) and treated them with CDT (FIG. 7B). Baseline TFR1 in BJAB was too high for TFRC introduction to increase it significantly (1.13× increase) causing no significant difference in CDT sensitivity. Farage cells, however, with 2.64× increased expression after TFRC introduction, became dramatically more sensitive to CDT, consistent with a TFR1-dependent mode of action. Multiple attempts to create stable RNAi TFRC knockdown clones of DLBCL lines were unsuccessful. Although this prevented assessment of TFR1 reduced expression effect on drug activity, it further illustrates the essential nature of the protein in DLBCL cells. As an alternate loss-of-function approach, flooding of TFR1 with its natural ligand holo-TF was utilized to reduce availability of binding by CDT. BJAB and Farage cells were co-incubated with CDT and the maximum non-toxic dose of Holo-TF (FIG. 8C). This caused a significant negative shift in CDT sensitivity in both lines (FIG. 7C). As a second alternate loss-of-function assessment, BJAB and Farage cells were co-incubated with CDT and the clathrin-mediated endocytosis inhibitor dynasore (47). Single agent dynasore at concentrations known to inhibit endocytosis (15 μM, 50 μM) negatively affected viability of BJAB and Farage cells somewhat (FIG. 7D, top panel). Normalized for baseline dynasore effect on viability, 100 nM CDT had greatly reduced efficacy in both lines (FIG. 7D, bottom panel). No noticeable change in TFR1 expression was found in response to CDT or non-toxic CND-TF treatment by 24-hours, consistent with rapid TFR1 recycling back to the cell surface, as previously described (48-50) even after exposure to toxic CDT (y FIG. 7D). To further investigate cellular events triggered upon CDT exposure, the inherent red fluorescence of CNDs and Dox with fluorescent confocal microscopy was used. While unconjugated CND and CND-Dox yielded little nuclear colocalization in BJAB, nuclear colocalization of CDT and Dox was similar, with CDT doing so at 10-fold lower concentration (FIG. 9). Because the large nucleus-to-cytoplasm ratio of lymphoma cells limits microscopic evaluation of intracellular events, embryonic kidney HEK293 cells were employed for further assessments. After confirming HEK293 cells were an appropriate system (FIG. 10A), cells were treated with CDT and Dox and repeated fluorescent confocal assessment. At a lower dose, it was determined that CDT entered the cytoplasm more efficiently, with significantly improved nuclear colocalization compared to Dox (FIG. 7E). Rapid nuclear Dox accumulation seems unlikely if it remains attached to the bulky TF protein. It was hypothesized that Dox, either alone or attached to the CND, separated from TF following TFR1-mediated endocytosis. To test this, CDT was synthesized using cyan-fluorophore tagged TF (CDTcy). After confirming similar potency of CDTcy compared to CDT (FIG. 10B), HEK293 cells were treated with CDTcy, staining the nucleus green to prevent fluorescence overlap (FIG. 7F). TFcy (blue) did not colocalize with red (Dox, CND) fluorescence in the nucleus, suggesting TF uncoupling in the cytoplasm prior to Dox reaching the nucleus. These data provide detailed insight to the mechanism of CDT therapy, demonstrating cellular entry through TFR1, subsequent loss of TF from the reagent and rapid entry of Dox into the nucleus.

### Example 5: Safe and Effective Dosing of CDT to PDX DLBCL-Bearing Mice

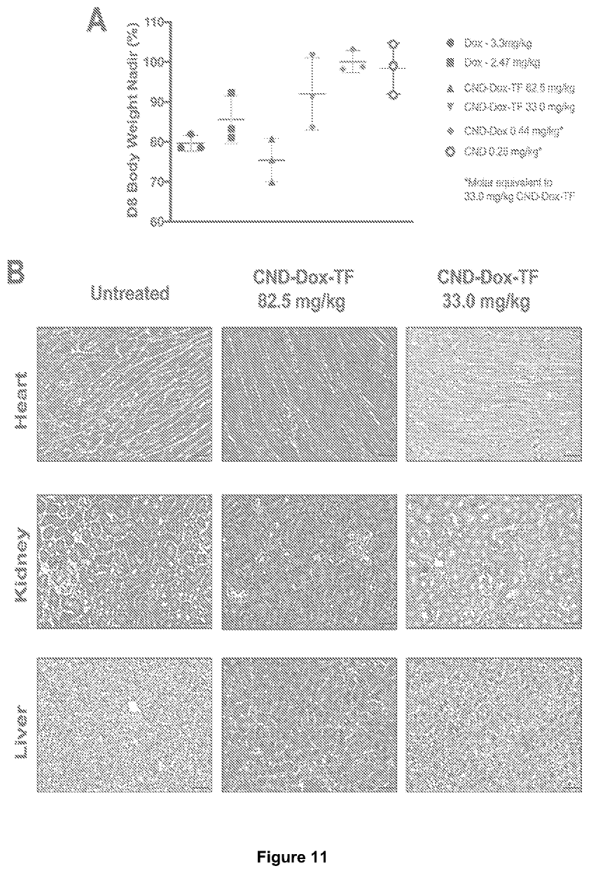

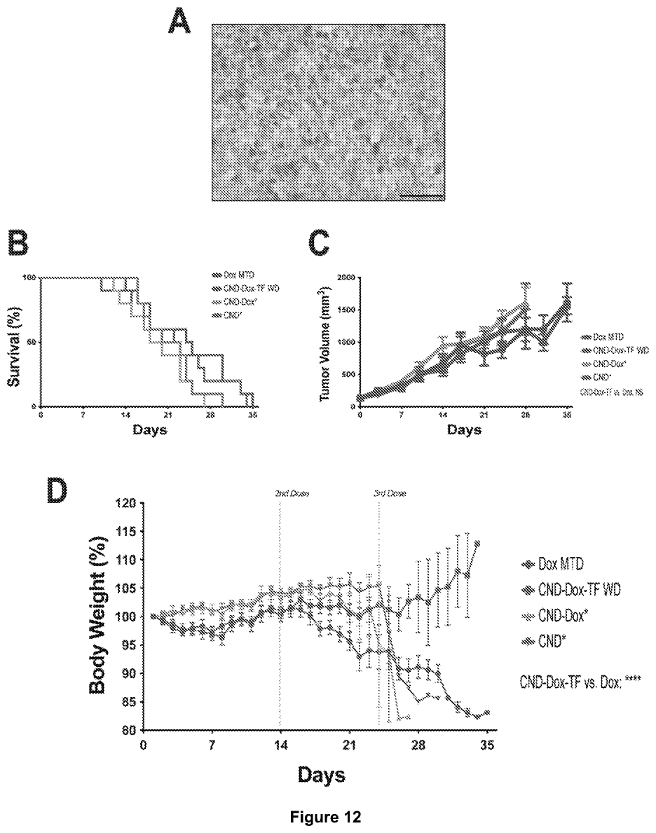

The safety and efficacy of CNDs were tested in NOD scid gamma (NSG) mice. While Dox at 2.47 mg/kg already had a significant effect on body weight, the maximum tolerated dose (MTD) of Dox was confirmed to be 3.3 mg/kg, leading to ˜20% weight loss (survival endpoint) (51-53). A single dose of 82.5 mg/kg of CDT in non-tumor bearing NSG mice was too toxic, while 33.0 mg/kg was well-tolerated (FIG. 11A). Importantly, dosing, equimolar to CDT 33.0 mg/kg, of CND-Dox (0.44 mg/kg) or CND (0.25 mg/kg) were non-toxic with no significant effects on body weight. Histology of heart, liver and kidney showed no necrosis from CDT treatment (FIG. 11B). CDT toxicity and efficacy in a DLBCL PDX model was assessed using the working dose (WD) of 33.0 mg/kg. PDX was established from a DLBCL tumor, germinal-center B-cell (GCB) subtype, excised from the spleen of a 57-year-old male with no prior history of treatment. Immunohistochemistry (IHC) analysis of the DLBCL PDX tumor confirmed TFR1 expression (FIG. 12A). NSG mice were engrafted with DLBCL PDX tumors and randomized at average tumor size 150 mm (3 to 4 groups of 10): MTD Dox, WD CDT, molar equivalent CND-Dox (0.44 mg/kg) or molar equivalent CND (0.25 mg/kg). Mice were treated i.v.×1 with each drug on days 0, 14, and 24. Treatment with CDT resulted in similar anti-tumor efficacy compared to Dox, as shown through OS and tumor volume assessments, with CND-Dox and CND having no activity (FIG. 12B-C). Treatment with Dox resulted in an expected continuous decline of body weight, with nadir by day 8, followed by a return to starting weight by day 10-14. CND-conjugate treatment to Dox treatment was mimicked as dictated by body weight changes with eventual return to starting weight triggering re-treatment. Both CDT- and Dox-treated mice followed this expected trend following the first dose, but after two additional doses, Dox treated mice experienced irreversible weight loss, while CDT treated mice did not (FIG. 12D). These initial in vivo studies showed CNDs can be safely administered in vivo. Importantly, a working dose of the full CDT therapy was identified that carried anti-lymphoma efficacy similar to single-agent Dox, while preliminarily appearing better tolerated. Since Dox is never used as a single agent clinically, relatively weak antitumor activity in this experiment was not unexpected but justified further evaluation as part of clinically relevant combination therapy.

### Example 6: R-nanoCHOP Prolongs Overall Survival Compared to R-CHOP in DLBCL PDX-Bearing Animals

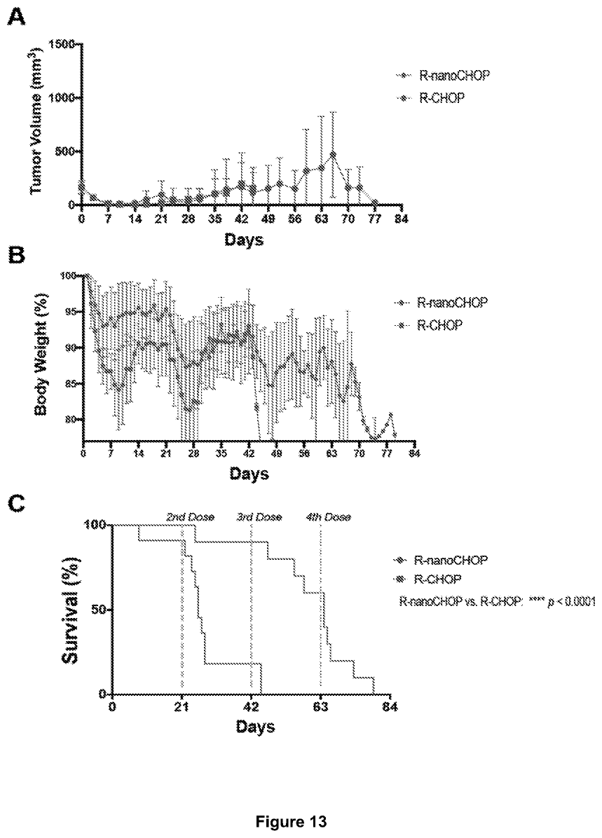

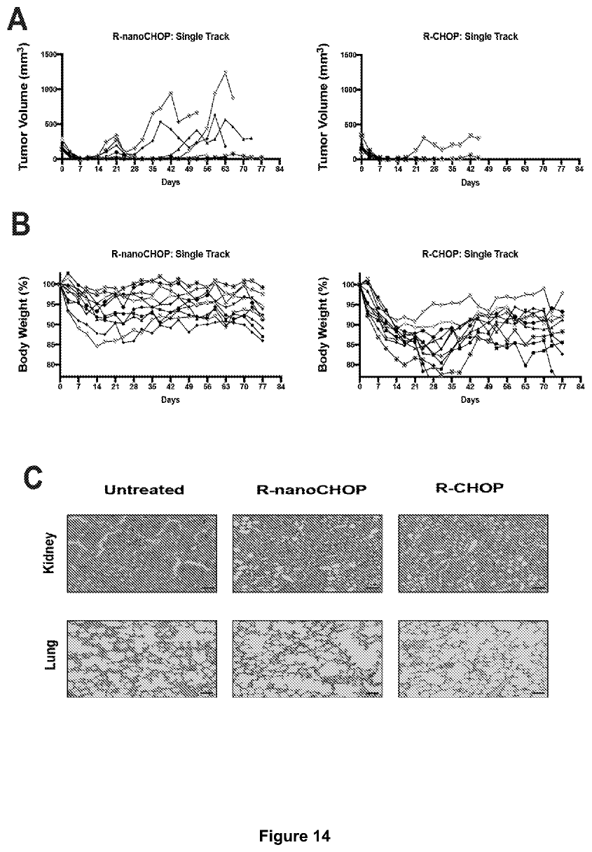

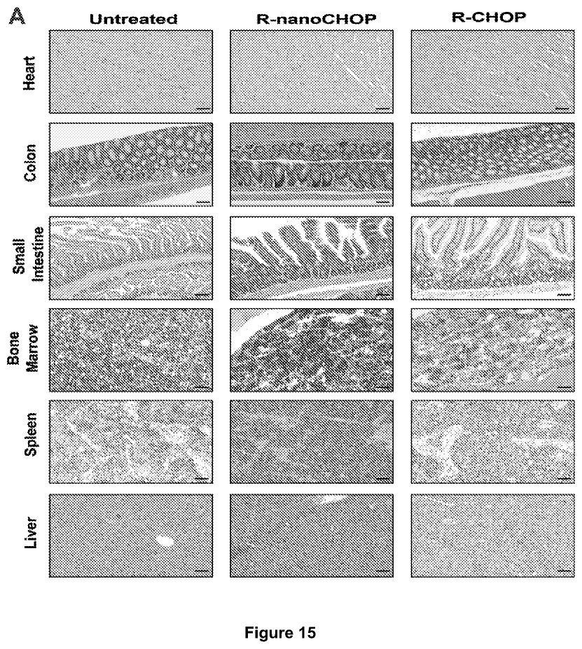

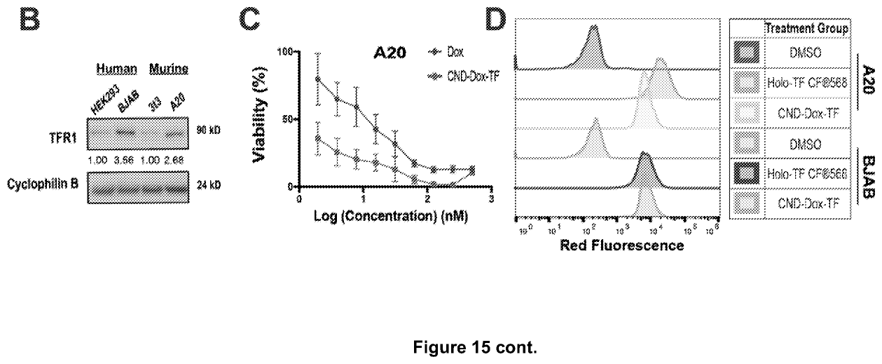

The 5-drug combination R-CHOP administered once on day 1 of every 21-day cycle is standard frontline therapy for DLBCL. In a clinically relevant assessment of the disclosed therapy, Dox in R-CHOP was replaced with CDT (R-nanoCHOP). Twenty-two (22) NSG mice were engrafted with DLBCL PDX tumors (FIG. 12A) and randomized to two groups of 11 at tumor engraftment for R-CHOP or R-nanoCHOP every 21 days. Of note, one mouse in the R-nanoCHOP group died prior to initiation of treatment and was excluded from further analysis. R-nanoCHOP (n=10) and R-CHOP (n=11) resulted in essentially identical tumor-volume control while both groups were alive (FIGS. 13A and 14A). R-nanoCHOP-treated mice, however, tolerated treatment with dramatically less weight loss than R-CHOP-treated animals (FIGS. 13B and 14B). This led to R-nanoCHOP-treated mice having significantly improved overall survival, tolerating an average of two additional treatment cycles compared to R-CHOP-treated mice (p<0.0001, FIG. 13C). Histology of R-nanoCHOP and R-CHOP treated mice showed minimal effects on vital organs of interest (FIGS. 15A and 14C). A decrease in cellularity in bone marrow and subtle evidence of hepatotoxicity, indicated by increased lobular inflammation, was seen in both treatment groups. Because animals were sacrificed by CO₂ euthanasia as required by ethical considerations at 20% weight loss, necroscopy did not identify specific therapy-related causes of death for mice in either group. The greatly reduced toxicity of CDT compared to Dox with at least equal antitumor activity was encouraging, but a potential caveat is the species difference between host and tumor in these experiments. To confirm CDT, containing human holo-TF, interacted similarly with murine and human TFR1, assessment of TFR1 expression using an antibody reactive to the protein from both species showed expression in the murine B-lymphoma line A20 similar to BJAB cells, while 3T3 non-transformed murine fibroblasts had lower expression (FIG. 15B). Like human DLBCL lines, A20 cells were dramatically more sensitive to CDT than to unconjugated Dox (FIG. 15C, CDT EC50 0.59 nM, Dox EC50 10.64 nM). This strongly suggests similar binding to murine and human TFR1 by CDT. For further confirmation, BJAB and A20 cells were exposed to 500 nM CDT or 25 ug/mL fluorophore-labeled human holo-TF (holo-TF CF®568) for 30 minutes and red fluorescence analyzed by flow cytometry (FIG. 15D). These results confirmed similar strong binding to both human BJAB and murine A20 cells by both reagents. In sum, human and murine TFR1 was bound similarly by human holo-TF, including as part of the CDT conjugate. The novel therapeutic regimen R-nanoCHOP has promising anti-lymphoma activity with a favorable toxicity profile that allowed administration of additional treatment cycles, prolonging overall survival.

### Example 7: Characeriziation of NanoDox-sc

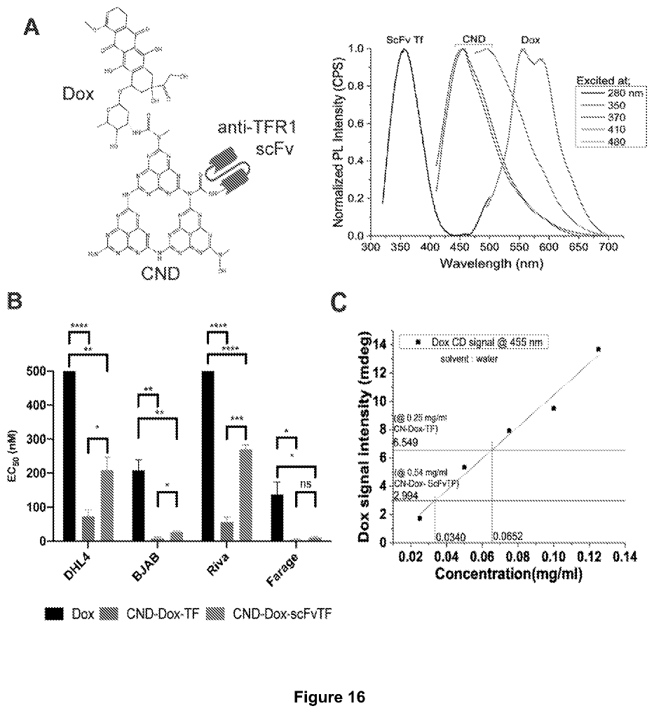

The full Holo-TF ligand was replaced with the murine IgG1 anti-human TFR1 single-chain variable fragment (scFv) 5E9 to create an initial version of NanoDox-sc (FIG. 16A). This resulted in an active molecule in initial testing against lymphoma cell lines (FIG. 16B). Reduced molar potency compared to NanoDox resulted from substantially reduced Dox loading in the initial synthesis of the scFv version: 1:2 CND-Dox ratio by circular dichroism compared to 1:18 for NanoDox (FIG. 16C). Initial activity of NanoDox-sc therefore was especially encouraging given it delivered one-nineth as many Dox molecules per mole of overall conjugate.

### SUMMARY

Frontline R-CHOP results in long-term disease-free survival in up to 60% of DLBCL, but salvage of rel/ref patients has limited success (1, 2). Recent advances in immunotherapy provide new options for subsets of patients, but costly and laborious ex vivo methodology, unfavorable clinical toxicities, and strict patient-eligibility requirements have hindered broad clinical implementation so far (54, 55). Overexpression of cell-surface receptor TFR1 is well described across cancer and has been therapeutically investigated in various solid-tumor malignancies (9-18). Here, TFR1 overexpression was linked to poor prognosis in DLBCL in two well-known large datasets from pretreatment biopsies of patients treated with standard therapies with curative intent (FIG. 1). TFR1-targeted therapy was therefore an opportunity to treat high-risk DLBCL tumors in a novel fashion.

Targeting TFR1 has been the focus of previously developed anti-cancer therapeutic compounds, either utilizing the receptor as an entry point to deliver toxic cargo, or simply blocking TFR1's growth-promoting capabilities through antagonistic antibodies or single-chain variable fragments (scFv) (57, 58). Pre-clinical testing of directly fused TF-Dox compounds revealed activity at the plasma membrane and cytoplasm, in strong contrast to unconjugated single-agent Dox's activity in the nucleus, and this discrepancy was among factors that ultimately halted further development (59-61). An engineered diphtheria toxin mutant CRM107 fused to TF promoted a tumor response in 9 of 15 evaluable brain-tumor patients during a phase 1 trial (24), but ultimately failed due to seizure toxicity of unclear etiology (25). TFR1-targeting nanoparticles successfully delivered siRNA to human melanoma patients but failed to evolve into a clinically applicable therapy (26). Recent advances in nanotechnology provide new opportunities to optimize the TFR1-targeted treatment paradigm. In addition, previous efforts have not assessed efficacy against DLBCL, a disease in which Dox is still considered the most active drug as part of frontline chemoimmunotherapy.

The CNDs disclosed herein are low-cost, non-toxic, eco-friendly with advantageous properties for therapeutic development (33, 36, 39, 40, 62, 63). As disclosed herein, the anti-tumor efficacy of a novel chemotherapeutic nanocarrier compound comprising holo-TF and Dox linked to CNDs was developed and tested (FIG. 3A). These results suggest this is a viable therapeutic strategy for targeting TFR1 in DLBCL. Cell viability assessments revealed DLBCL cell lines are dramatically more sensitive to CDT than single-agent Dox (FIGS. 6A and 6B), with onset of apoptosis and DNA damage occurring at lower doses (FIGS. 5C and 5D). Overall, in cell lines, baseline sensitivity to Dox appears most closely associated with observed CDT efficacy rather than the absolute level of TFR1 expression. This is consistent with well-described rapid recycling of TFR1 back to the cell surface after each round of endocytosis (48-50), and so while raw TFR1-expression may differ across cell lines, rapid CDT uptake occurs nonetheless (FIG. 7D). Regardless, gain-and-loss of function experiments demonstrate CDT activity is ultimately mediated by TFR1 (FIGS. 7A-7D) and facilitated cellular entry via TF-TFR1 interaction (FIG. 7E) with subsequent cytoplasmic separation of Dox, either alone or still bound to CND, from TF (FIG. 7F). This enabled strong entry and activity of Dox in the nucleus. The safety of CNDs in NSG mice were established and a working dose identified for CDT (FIGS. 11 and 12).

When compared with Dox MTD in a high-TFR1 expressing DLBCL PDX model, CDT has similar anti-tumor efficacy and an improved toxicity profile (FIG. 12). To more accurately explore clinical relevance, Dox was substituted with CDT in frontline R-CHOP, creating R-nanoCHOP, administered in clinically standard 21-day cycles. Compared with R-CHOP, R-nanoCHOP had similar anti-lymphoma efficacy with diminished toxicity (FIGS. 13A and 13B). Strikingly, a significant improvement was observed in the OS of R-nanoCHOP treated mice compared to those treated with R-CHOP (FIG. 13C). Histology showed similar effects of both treatment modalities; however this level of minimal toxicity occurred in R-nanoCHOP treated mice on average two cycles after it was seen in R-CHOP treated mice. CDT has similar activity against and binding to human and murine cells, addressing potential caveats regarding possible lack of cross-species reactivity (FIGS. 15B-15D). This shows R-nanoCHOP delayed non-malignant organ toxicity substantially longer than R-CHOP (FIGS. 14 and 15A). Specifically, the indication of diminished negative effects was observed in the bone marrow with R-nanoCHOP, despite known high TFR1 expression on cells of the erythrocyte lineage. Additionally, in spite of additional dosing with R-nanoCHOP, no increase in cardiotoxicity was observed. CDTsc, a synthesized version in which holo-TF is replaced with an anti-TF scFv. The resulting substantial decrease in molecular weight (>50% overall) can be expected to improve pharmacokinetic properties and allow increased dosing, while preserving strong anti-lymphoma activities. This system is also highly adaptable to conjugation of additional antineoplastic compounds and optimization with modifications that enhance delivery inside cells such as linkers specifically cleaved in lysosomes as are used in approved antibody-drug conjugates (65).

The examples herein indicate an association between TFR1 expression and reduced survival of DLBCL patients, pointing to TFR1 as a novel target to improve outcomes for high-risk cases. CND-Dox-TF (CDT), a novel reagent for targeted CND-based delivery of Dox to tumors exploiting TFR1-mediated endocytosis is further disclosed. CDT dramatically increased potency against DLBCL cell lines compared to Dox. In vivo, replacement of Dox with CDT in R-CHOP (R-nanoCHOP) significantly improved survival of NOD scid gamma (NSG) mice bearing DLBCL PDX tumors. CDT's successful delivery of Dox to DLBCL tumors in vivo with diminished treatment-limiting off-tumor toxicities demonstrated a proof of principle for targeting TFR1 in this disease. These studies provide a compelling rationale for development of TFR1-targeting therapies for DLBCL. The use of optimized reagents based on the CDT concept in place of Dox could improve DLBCL patient outcomes in frontline or rel/ref settings.

### SIGNIFICANCE

TFR1 identifies high-risk cases of DLBCL. Targeted nanoparticle delivery of doxorubicin chemotherapy turns the receptor into a new opportunity to target these tumors with potency and precision.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims

### REFERENCES

1. Siegel, R. L., K. D. Miller, and A. Jemal, Cancer statistics, 2019. CA Cancer J Clin, 2019. 69(1): p. 7-34.
2. Friedberg, J. W., Relapsed/refractory diffuse large B-cell lymphoma. Hematology Am Soc Hematol Educ Program, 2011. 2011: p. 498-505.
3. Bonadonna, G., et al., Clinical evaluation of adriamycin, a new antitumour antibiotic. Br Med J, 1969. 3(5669): p. 503-6.
4. Chatterjee, K., et al., Doxorubicin cardiomyopathy. Cardiology, 2010. 115(2): p. 155-62.
5. Zhao, L. and B. Zhang, Doxorubicin induces cardiotoxicity through upregulation of death receptors mediated apoptosis in cardiomyocytes. Sci Rep, 2017. 7: p. 44735.
6. Myers, C., The role of iron in doxorubicin-induced cardiomyopathy. Semin Oncol, 1998. 25(4 Suppl 10): p. 10-4.
7. Daniels, T. R., et al., The transferrin receptor part I: Biology and targeting with cytotoxic antibodies for the treatment of cancer. Clin Immunol, 2006. 121(2): p. 144-58.
8. Torti, S. V. and F. M. Torti, Iron and cancer: more ore to be mined. Nat Rev Cancer, 2013. 13(5): p. 342-55.
9. Daniels, T. R., et al., The transferrin receptor part II: targeted delivery of therapeutic agents into cancer cells. Clin Immunol, 2006. 121(2): p. 159-76.
10. Yang, D. C., et al., Expression of transferrin receptor and ferritin H-chain mRNA are associated with clinical and histopathological prognostic indicators in breast cancer. Anticancer Res, 2001. 21(1B): p. 541-9.
11. Prior, R., G. Reifenberger, and W. Wechsler, Transferrin receptor expression in tumours of the human nervous system: relation to tumour type, grading and tumour growth fraction. Virchows Arch A Pathol Anat Histopathol, 1990. 416(6): p. 491-6.
12. Kondo, K., et al., Transferrin receptor expression in adenocarcinoma of the lung as a histopathologic indicator of prognosis. Chest, 1990. 97(6): p. 1367-71.
13. Seymour, G. J., et al., Transferrin receptor expression by human bladder transitional cell carcinomas. Urol Res, 1987. 15(6): p. 341-4.
14. Wu, H., et al., Transferrin receptor-1 and VEGF are prognostic factors for osteosarcoma. J Orthop Surg Res, 2019. 14(1): p. 296.
15. Sakurai, K., et al., Immunohistochemical demonstration of transferrin receptor 1 and 2 in human hepatocellular carcinoma tissue. Hepatogastroenterology, 2014. 61(130): p. 426-30.
16. Rosager, A. M., et al., Transferrin receptor-1 and ferritin heavy and light chains in astrocytic brain tumors: Expression and prognostic value. PLoS One, 2017. 12(8): p. e0182954.
17. Habashy, H. O., et al., Transferrin receptor (CD71) is a marker of poor prognosis in breast cancer and can predict response to tamoxifen. Breast Cancer Res Treat, 2010. 119(2): p. 283-93.
18. Basuli, D., et al., Iron addiction: a novel therapeutic target in ovarian cancer. Oncogene, 2017. 36(29): p. 4089-4099.
19. Xu, L., et al., Systemic p53 gene therapy of cancer with immunolipoplexes targeted by anti-transferrin receptor scFv. Mol Med, 2001. 7(10): p. 723-34.
20. Xu, L., et al., Systemic tumor-targeted gene delivery by anti-transferrin receptor scFv-immunoliposomes. Mol Cancer Ther, 2002. 1(5): p. 337-46.
21. Yu, W., et al., A sterically stabilized immunolipoplex for systemic administration of a therapeutic gene. Gene Ther, 2004. 11(19): p. 1434-40.
22. Senzer, N., et al., Phase I study of a systemically delivered p53 nanoparticle in advanced solid tumors. Mol Ther, 2013. 21(5): p. 1096-103.
23. Pirollo, K. F., et al., Safety and Efficacy in Advanced Solid Tumors of a Targeted Nanocomplex Carrying the p53 Gene Used in Combination with Docetaxel: A Phase 1b Study. Mol Ther, 2016. 24(9): p. 1697-706.
24. Laske, D. W., R. J. Youle, and E. H. Oldfield, Tumor regression with regional distribution of the targeted toxin TF-CRM107 in patients with malignant brain tumors. Nat Med, 1997. 3(12): p. 1362-8.
25. Weaver, M. and D. W. Laske, Transferrin receptor ligand-targeted toxin conjugate (Tf-CRM107) for therapy of malignant gliomas. J Neurooncol, 2003. 65(1): p. 3-13.
26. Davis, M. E., et al., Evidence of RNAi in humans from systemically administered siRNA via targeted nanoparticles. Nature, 2010. 464(7291): p. 1067-70.
27. Li, S., et al., “Dark” carbon dots specifically “light-up” calcified zebrafish bones. J Mater Chem B, 2016. 4(46): p. 7398-7405.
28. Wang, W., et al., Facile synthesis of water-soluble and biocompatible fluorescent nitrogen-doped carbon dots for cell imaging. Analyst, 2014. 139(7): p. 1692-1696.
29. Ma, J., et al., One-pot fabrication of hollow cross-linked fluorescent carbon nitride nanoparticles and their application in the detection of mercuric ions. Talanta, 2015. 143: p. 205-211.
30. Zhou, J., Y. Yang, and C.-y. Zhang, A low-temperature solid-phase method to synthesize highly fluorescent carbon nitride dots with tunable emission. Chemical Communications, 2013. 49(77): p. 8605-8607.
31. Ge, J., et al., Red-Emissive Carbon Dots for Fluorescent, Photoacoustic, and Thermal Theranostics in Living Mice. Adv Mater, 2015. 27(28): p. 4169-77.
32. Jiang, K., et al., Red, green, and blue luminescence by carbon dots: full-color emission tuning and multicolor cellular imaging. Angew Chem Int Ed Engl, 2015. 54(18): p. 5360-3.
33. Peng, Z., et al., Carbon dots: promising biomaterials for bone-specific imaging and drug delivery. Nanoscale, 2017. 9(44): p. 17533-17543.
34. Yang, L., et al., One pot synthesis of highly luminescent polyethylene glycol anchored carbon dots functionalized with a nuclear localization signal peptide for cell nucleus imaging. Nanoscale, 2015. 7(14): p. 6104-13.
35. Huang, X., et al., Effect of injection routes on the biodistribution, clearance, and tumor uptake of carbon dots. ACS Nano, 2013. 7(7): p. 5684-93.
36. Ruan, S., et al., A simple one-step method to prepare fluorescent carbon dots and their potential application in non-invasive glioma imaging. Nanoscale, 2014. 6(17): p. 10040-7.
37. Yang, S. T., et al., Carbon dots for optical imaging in vivo. J Am Chem Soc, 2009. 131(32): p. 11308-9.
38. Zheng, M., et al., Self-Targeting Fluorescent Carbon Dots for Diagnosis of Brain Cancer Cells. ACS Nano, 2015. 9(11): p. 11455-61.
39. Liyanage, P. Y., et al., Carbon Nitride Dots: A Selective Bioimaging Nanomaterial. Bioconjug Chem, 2019. 30(1): p. 111-123.
40. Liyanage, P. Y., et al., Pediatric glioblastoma target-specific efficient delivery of gemcitabine across the blood-brain barrier via carbon nitride dots. Nanoscale, 2020.
41. Hettiarachchi, S. D., et al., Triple conjugated carbon dots as a nano-drug delivery model for glioblastoma brain tumors. Nanoscale, 2019. 11(13): p. 6192-6205.
42. Aguirre-Gamboa, R., et al., SurvExpress: an online biomarker validation tool and database for cancer gene expression data using survival analysis. PLoS One, 2013. 8(9): p. e74250.
43. Lenz, G., et al., Stromal gene signatures in large-B-cell lymphomas. N Engl J Med, 2008. 359(22): p. 2313-23.
44. Reddy, A., et al., Genetic and Functional Drivers of Diffuse Large B Cell Lymphoma. Cell, 2017. 171(2): p. 481-494 e15.
45. Binaschi, M., et al., Anthracyclines: selected new developments. Curr Med Chem Anticancer Agents, 2001. 1(2): p. 113-30.
46. Tacar, O., P. Sriamornsak, and C. R. Dass, Doxorubicin: an update on anticancer molecular action, toxicity and novel drug delivery systems. J Pharm Pharmacol, 2013. 65(2): p. 157-70.
47. Macia, E., et al., Dynasore, a cell-permeable inhibitor of dynamin. Dev Cell, 2006. 10(6): p. 839-50.
48. Mayor, S., J. F. Presley, and F. R. Maxfield, Sorting of membrane components from endosomes and subsequent recycling to the cell surface occurs by a bulk flow process. J Cell Biol, 1993. 121(6): p. 1257-69.
49. Ciechanover, A., et al., Kinetics of internalization and recycling of transferrin and the transferrin receptor in a human hepatoma cell line. Effect of lysosomotropic agents. J Biol Chem, 1983. 258(16): p. 9681-9.
50. Maxfield, F. R. and T. E. McGraw, Endocytic recycling. Nat Rev Mol Cell Biol, 2004. 5(2): p. 121-32.
51. Mohammad, R. M., et al., Genistein sensitizes diffuse large cell lymphoma to CHOP (cyclophosphamide, doxorubicin, vincristine, prednisone) chemotherapy. Mol Cancer Ther, 2003. 2(12): p. 1361-8.
52. Mohammad, R. M., et al., The addition of bryostatin 1 to cyclophosphamide, doxorubicin, vincristine, and prednisone (CHOP) chemotherapy improves response in a CHOP-resistant human diffuse large cell lymphoma xenograft model. Clin Cancer Res, 2000. 6(12): p. 4950-6.
53. Al-Katib, A., et al., I-kappa-kinase-2 (IKK-2) inhibition potentiates vincristine cytotoxicity in non-Hodgkin's lymphoma. Mol Cancer, 2010. 9: p. 228.
54. Schuster, S. J., et al., Tisagenlecleucel in Adult Relapsed or Refractory Diffuse Large B-Cell Lymphoma. N Engl J Med, 2019. 380(1): p. 45-56.
55. Lin, J. K., et al., Cost Effectiveness of Chimeric Antigen Receptor T-Cell Therapy in Multiply Relapsed or Refractory Adult Large B-Cell Lymphoma. J Clin Oncol, 2019. 37(24): p. 2105-2119.
56. Habeshaw, J. A., et al., Correlation of transferrin receptor expression with histological class and outcome in non-Hodgkin lymphoma. Lancet, 1983. 1(8323): p. 498-501.
57. Brooks, D., et al., Phase la trial of murine immunoglobulin A antitransferrin receptor antibody 42/6. Clin Cancer Res, 1995. 1(11): p. 1259-65.
58. Crepin, R., et al., Development of human single-chain antibodies to the transferrin receptor that effectively antagonize the growth of leukemias and lymphomas. Cancer Res, 2010. 70(13): p. 5497-506.
59. Barabas, K., J. A. Sizensky, and W. P. Faulk, Transferrin conjugates of adriamycin are cytotoxic without intercalating nuclear DNA. J Biol Chem, 1992. 267(13): p. 9437-42.
60. Kratz, F., et al., Transferrin conjugates of doxorubicin: synthesis, characterization, cellular uptake, and in vitro efficacy. J Pharm Sci, 1998. 87(3): p. 338-46.
61. Lai, B. T., J. P. Gao, and K. W. Lanks, Mechanism of action and spectrum of cell lines sensitive to a doxorubicin-transferrin conjugate. Cancer Chemother Pharmacol, 1998. 41(2): p. 155-60.
62. Bao, X., et al., In vivo theranostics with near-infrared-emitting carbon dots-highly efficient photothermal therapy based on passive targeting after intravenous administration. Light Sci Appl, 2018. 7: p. 91.
63. Feng, T., et al., Charge-Convertible Carbon Dots for Imaging-Guided Drug Delivery with Enhanced in Vivo Cancer Therapeutic Efficiency. ACS Nano, 2016. 10(4): p. 4410-20.
64. Favreau-Lessard, A. J., et al., Systemic and cardiac susceptibility of immune compromised mice to doxorubicin. Cardiooncology, 2019. 5: p. 2.
65. Younes, A., et al., Brentuximab vedotin (SGN-35) for relapsed CD30-positive lymphomas. N Engl J Med, 2010. 363(19): p. 1812-21.

1: A therapeutic composition comprising a carbon-nitride dot nanocarrier having a surface comprising carbodiimide cross-linked doxorubicin and transferrin thereupon.

2: A therapeutic composition comprising rituximab, cyclophosphamide, vincristine, prednisone, transferrin, and doxorubicin, wherein the doxorubicin and transferrin is a carbodiimide cross-linked doxorubicin and transferrin on the surface of a carbon-nitride dot nanocarrier.

3: The composition of claim 2, wherein the nanocarrier contains triazine rings (C₃N₄).

4: The composition of claim 2, wherein the nanocarrier is synthesized from urea and citric acid.

5: The composition of claim 2, wherein the nanocarrier contains amine groups, amide groups, and carboxyl groups on its surface.

6: The composition of claim 2, wherein the nanocarrier has an excitation wavelength between 450-600 nm.

7: The composition of claim 2, having a potency against diffuse large B-cell lymphoma in vitro that is 10-100 fold greater than doxorubicin treatment alone.

8: The composition of claim 2, having an LD₅₀ to diffuse large B-cell lymphoma that is less than 100 nm.

9: The composition of 2, wherein the carbodiimide cross-linked doxorubicin and transferrin have increased in vivo efficacy.

10: A method of treating cancer, in a subject in need thereof, comprising administering the composition of claim 2 to an individual having solid tumor cells overexpressing transferrin receptor (TFR1).

11: A method of treating cancer, in a subject in need thereof, comprising administering the composition of claim 2 to an individual with a blood or circulating cancer overexpressing transferrin receptor (TFR1).

12: The method of claim 11, wherein the blood or circulating cancer is lymphoma.

13: The method of claim 11, wherein the composition increases the anti-lymphoma efficacy of doxorubicin on diffuse large B-cell lymphoma cell lines.

14: A therapeutic composition comprising cross-linked doxorubicin and transferrin on a carbon-nitride dot nanocarrier, wherein the nanocarrier is conjugated with a single-chain variable fragment (scFv) against transferrin receptor 1.

15: A therapeutic composition comprising, rituximab, cyclophosphamide, doxorubicin, vincristine and prednisone, wherein the doxorubicin is cross-linked doxorubicin of claim 14.
